(19) United States
(12) Patent Application Publication
(10) Pub. No.: US 2005/0282239 A1
(43) Pub. Date: Dec. 22, 2005

(54) Cell-permeable enzyme activation reporter that can be loaded in a high throughput and gentle manner

(76) Inventors: Nancy L. Allbritton, Irvine, CA (US); Christopher E. Sims, Irvine, CA (US); Francis M. Rossi, Menlo Park, CA (US); Joseph S. Soughayer, Portland, OR (US)

Correspondence Address: EVAN LAW GROUP LLC, 566 WEST ADAMS, SUITE 350, CHICAGO, IL, 60661, US

(21) Appl. No.: 11/016,542

(22) Filed: Dec. 17, 2004

Related U.S. Application Data
(60) Provisional application No. 60/530,875, filed on Dec. 17, 2003.

(51) Int. Cl. C12Q 1/48; C07K 14/47

(52) U.S. Cl. 435/15; 530/409

(57) ABSTRACT

A membrane traversing peptide conjugate comprises (i) a label, (ii) a target peptide, attached to the label, and (iii) a transduction domain, attached to the target peptide. The target peptide is a reactant for a chemical reaction occurring in a cell.

This application claims the benefit of U.S. Provisional Application No. 60/530,875 filed 17 Dec. 2003.

### STATEMENT OF ACKNOWLEDGMENT OF GOVERNMENT SUPPORT

This invention was made with Government support under Grant Nos. CA91216, GM57015 and NS39310, awarded by the National Institutes of Health. The Government has certain rights in this invention.

### BACKGROUND

The body prevents a number of defenses against outside invaders, which manifest in tissues, organs and systems. The skin sets up a passive first line of defense, for example, while the immune system actively assails those invaders that have breached other barriers. But even cells themselves—regardless of their type—erect a barrier which grants them control over cellular entry: the cell membrane. For the most part, only by duping cell-surface molecules or by physical disruption may invaders (e.g., molecules, viruses, bacteria) gain entry.

While cell membranes form a desirable barrier that prevents the flux of most molecules into and out of cells (Lipinski, Lombardo et al. 2001), this same fortification presents challenges to those exogenous molecules that may exert beneficial effects, such as cell membrane-impermeant pharmaceuticals. Furthermore, the study of cellular mechanisms and diagnosing diseases and disorders are also hampered. Nevertheless, the introduction of polar, membrane-impermeant molecules into cells has played an important role in the investigation of cellular behavior (Stein, Weiss et al. 1999; Stolzenberger, Haake et al. 2001; Cao, Pei et al. 2002; Ribeiro, Klein et al. 2003; Shibagaki and Udey 2003). The introduction of these molecules, however, have not been without significant cost, both to the target cells as well as to the proper understanding of data resulting from these molecular perturbations.

Attempts to Physically Breach the Cell Membrane

Exogenous molecules have been introduced into cells by microinjection, optoporation, pinocytosis and electroporation. Microinjection involves delicately inserting a pi pet into the cell and pressure-injecting a highly-concentrated solution into the cell. Optoporation requires aiming a pulsed, high-powered laser near a cell of interest to generate a compression wave with enough energy to permeabilize the cellular membrane. Pinocytosis, a normal cellular process, can be artificially induced by subjecting the cell to dramatic changes in osmolarity (ion concentrations). Electroporation subjects the target cell to Frankensteinian high-powered electric fields.

The disadvantages of these methods match the intimidating terminology. The primary disadvantage of each is that the target cells are severely stressed, resulting in aberrant activation of intracellular proteins. In all of these methods but pinocytosis, physical holes are poked into the cell membranes, disrupting membrane integrity. Microinjection and optoporation have the added disadvantages in that they can only be performed on only one cell (or few cells) at a time, severely limiting their usefulness. These methods also require expensive, dedicated equipment, and in many cases, large amounts of the membrane-impermeant molecules.

Various methods have been developed for delivering macromolecules into cells in vitro. A list of such methods includes electroporation, membrane fusion with liposomes, high velocity bombardment with DNA-coated microprojectiles, incubation with calcium-phosphate-DNA precipitate, DEAE-dextran mediated transfection, infection with modified viral nucleic acids, and direct micro-injection into single cells. These in vitro methods typically deliver the nucleic acid molecules into only a fraction of the total cell population, and they tend to damage large numbers of cells. Experimental delivery of macromolecules into cells in vivo has been accomplished with scrape loading, calcium phosphate precipitates and liposomes. However, these techniques have, to date, shown limited usefulness for in vivo cellular delivery. Moreover, even with cells in vitro, such methods are of extremely limited usefulness for delivery of proteins.

General methods for efficient delivery of biologically active proteins into intact cells, both in vitro and in vivo, are needed. (Sternson 1987) Chemical addition of a lipopeptide (Hoffmann 1988) or a basic polymer such as polylysine or polyarginine (Chen et al. 1978) have not proved to be highly reliable or generally useful (see Example 4 infra). Folic acid has been used as a transport moiety (Leamon and Low 1991). Evidence was presented for internalization of folate conjugates, but not for cytoplasmic delivery. Given the high levels of circulating folate in vivo, the usefulness of this system has not been fully demonstrated. Pseudomonas exotoxin has also been used as a transport moiety (Prior 1991). The efficiency and general applicability of this system for the intracellular delivery of biologically active cargo molecules is not clear from the published work, however.

Attempts to Breach the Cell Membrane Using Fusion Polypeptides

The concept of exploiting a part of a polypeptide to breach cell membranes has been attempted by others, with mixed results. For example, polymers composed of contiguous, highly basic subunits, particularly subunits containing guanidyl or amidinyl moieties, when conjugated to small molecules or macromolecules are effective in enhancing transport of the attached molecule across biological membranes (Rothbard and Wender 2001). This same reference purports to have a more efficient transport rate than that provided by the basic HIV TAT peptide (see below). Others have tried, for example, to rectify the main characteristic of some molecules that prevent transport: a overall negative charge (Ryser and Shen 1989). In this approach, the negative charge is cancelled out by covalently attaching to the molecule highly positively charged polymers (e.g., primary amines). In both of these approaches, however, the confounding effects of the membrane breaching-enabling molecules cannot be discounted once the cargo molecules have entered the cell, although Rothbard et al. (2001) suggest using a cleavable linkage between the cargo and membrane breaching-enabling molecule.

Protein Transduction Domains

Short peptide sequences known as protein transduction domains (PTDs; also known as cell-penetrating peptides), have become increasingly prevalent as tools to internalize otherwise impermeant molecules into cells (Becker-Hapak, McAllister et al. 2001). Of the numerous PTDs (Table 1), the transduction domain from the Human Immunodeficiency Virus (HIV) TAT protein (GenBank Accession NP_{—}057853; SEQ ID NO:1), a transcription activator, has been widely used and characterized (Lindsay 2002; Wadia and Dowdy 2002). Exogenously delivered TAT protein can translocate to the nucleus and activate transcription (Frankel and Pabo 1988; Green and Loewenstein 1988).

TABLE 1

Protein translocation peptides
(Lindgren, Hallbrink et al. 2000)*

| | | |
|---|---|---|
| Molecule | Sequence | SEQ ID NO: |

| | | |
|---|---|---|
| Penetratin | RQIKIWFQNRRMKWKK | 3 |
| Tat fragment | GRKKRRQRRRPPQ | 4 |
| (48-60) | | |
| Signal- | GALFLGWLGAAGSTMGAWSQ | 5 |
| sequence-based | PKKKRKV | |
| peptides (I) | | |
| Signal- | AAVALLPAVLLALLAP | 6 |
| sequence-based | | |
| peptides (II) | | |
| Transportan | GWTLNSAGYLLKINLKALAAL | 7 |
| | AKKIL | |
| Amphiphilic | KLALKLALKALKAALKLA | 8 |
| model peptide | | |
| Antp protein | GenBank Accession | 9 |
| from D. | NP_788590 | |
| melanogaster | | |
| HSV VP22 | GenBank Accession | 10 |
| | NP_044511 | |

TAT protein, about 86-102 amino acids (depending on the viral strain) (Lindgren, Hallbrink et al. 2000) is involved in HIV replication. Three domains are evident, both structurally and functionally: (1) the N-terminus, which is highly acidic and responsible for transcription activation; (2) a classic DNA-binding zinc (Zn) finger motif in a cysteine rich region (approximately 22-37 residues); and (3) a basic region which mediates nuclear import (49-58 residues); this region is though to play a role in Ca²⁺-independent attachment to cell membranes (Lindgren, Hallbrink et al. 2000).

The TAT protein's ability to cross the plasma membrane has since been resolved to a highly basic region of 9 amino acid residues (RKKRRQRRR; SEQ ID NO:2; residues 49-57; hereinafter “TAT₍₄₉₋₅₇₎”) (Vogel, Lee et al. 1993; Wender, Mitchell et al. 2000). Polypeptides incorporating this sequence have been used to deliver small peptides and peptide nucleic acids to full-length proteins and nanoparticles into cells (Schwarze, Ho et al. 1999; Lewin, Carlesso et al. 2000; Richard, Melikov et al. 2003).

The exact mechanism by which TAT enters cells is unknown. Transduction may be mediated by a novel pathway of transport since some work suggests that PTD entry is receptor-independent and occurs in essentially all cell types (Derossi, Calvet et al. 1996; Schwarze, Ho et al. 1999). Efficient intracellular uptake of the TAT peptide at 4° C. suggests that uptake occurs by a process other than endocytosis (which essentially does not occur in chilled cells) (Vives, Brodin et al. 1997; Futaki, Suzuki et al. 2001); pharmacologic inhibitors to block active cellular uptake processes such as endocytosis confirm the passive nature of TAT cellular entry (Suzuki, Futaki et al. 2002).

However, these studies may be confounded by the observation that due to TAT's high affinity for the plasma membrane, TAT conjugates bind to cell membranes even at low temperatures and remain after washing. Once bound, TAT conjugates are susceptible to being actively taken up by the cells (e.g., endocytosis) when chilled cells are returned to physiological temperatures (Richard, Melikov et al. 2003). To counter this artifact, the protease trypsin has been used in wash steps to remove bound TAT conjugates from the cell surface before analysis (Fittipaldi, Ferrari et al. 2003; Richard, Melikov et al. 2003). Contrary to previous studies suggesting that endocytosis does not play a role in TAT uptake, lipid rafts and caveolar endocytosis have been implicated, as well as being energy (ATP)-dependent (Fittipaldi, Ferrari et al. 2003; Richard, Melikov et al. 2003).

While TAT can breach the cell membrane barrier, too much TAT (concentrations greater than 5 μM) often kills cells. Unfortunately, toxic concentrations are necessary to introduce sufficient concentrations of some reporter molecules, such as fluorescently-tagged TAT (Vives, Brodin et al. 1997; Hallbrink, Floren et al. 2001). The ability to exploit TAT-mediated entry into cells at low concentrations would be valuable in not only studies of cellular behavior, but also in the delivery of pharmaceuticals.

Previous studies covalently attach TAT to the cargo molecules. Once in the cell, however, the cargo no longer requires TAT's assistance; however, its continued linkage to the cargo may ultimately inhibit the cargo's intended activity (Stein, Weiss et al. 1999; Astriab-Fisher, Sergueev et al. 2002; Kelemen, Hsiao et al. 2002). Even the bioactivity of an anti-tetanus F(ab′)2 fragments was inhibited unless transported into cells conjugated to a TAT peptide via a cleavable disulfide bond (Stein, Weiss et al. 1999).

Cells have erected formidable physical barriers, their cell membranes. Methods to breach the membrane either leave the cells for dead, functioning in exceptional ways (e.g., manifesting a stress response), or interacting with molecules which functions are often reduced or inhibited by the modification that enabled the molecule to penetrate the cell membrane in the first place.

### SUMMARY OF THE INVENTION

In a first aspect, the present invention is a membrane traversing peptide conjugate, comprising (i) a label, (ii) a target peptide, attached to the label, and (iii) a transduction domain, attached to the target peptide. The target peptide is a reactant for a chemical reaction occurring in a cell.

In a second aspect, the present invention is a membrane traversing peptide conjugate, comprising (i) a label, (ii) a target peptide, attached to the label, and (iii) a transduction domain, attached to the target peptide. The target peptide is a reactant for a chemical reaction occurring in a cell, the label is fluorescent, the target peptide is a kinase substrate, and the cell is a human cell.

In a third aspect, the present invention is a membrane traversing peptide conjugate, comprising (i) a label, (ii) an enzyme substrate, attached to the label, and (iii) a transduction domain, attached to the enzyme substrate. Thee label is a radioactive element, a fluorescent molecule, a phosphorescent molecule, a luminescent molecule, or a chemiluminescent molecule.

### DESCRIPTION OF THE FIGURES

FIG. 1 depicts use of sulfide/maleimide chemistry to conjugate target peptide and PTD to form a membrane traversing peptide conjugate;

FIG. 2 depicts intracellular cleavage of the membrane traversing peptide conjugate;

FIG. 3 depicts photochemical cleavage of the membrane transversing peptide conjugate;

FIG. 4 depicts a general scheme for preparing membrane traversing peptide conjugates containing a photolabile linkage and its subsequent cleavage using light;

FIG. 5 depicts electrophoretic analysis of single cells loaded with peptide substrates conjugated to TAT (49-57) through a disulfide bond. A) Electrophoretic trace of a mixture of F-CamKII (9×10⁻²⁰ mol) and phosphorylated F-CamKII (5×10⁻¹⁹ mol) in buffer solution. B) HT1080 cell loaded with the disulfide conjugate F-CamKII-C-SS-TAT. Shown is a typical electrophoretic trace from a single-cell analysis. C) Electrophoretic trace of a mixture of F-PKB (8×10⁻²⁰ mol) and phosphorylated F-PKB (1×10⁻¹⁹ mol) in buffer solution. D) Electrophoretic trace of an HT1080/PTEN cell loaded with the disulfide conjugate F-PKB-C-SS-TAT at an extracellular concentration of 1 μM. E) Electrophoretic trace of an HT1080/PTEN cell loaded as in “D” but demonstrating a multitude of unidentified peaks;

FIG. 6 shows analysis of phosphorylation of the PKB substrate in HT1080 and RBL cells. Electrophoretic traces from cells loaded with F-PKB-C-SS-TAT (1 μM). A single asterisk denotes the migration time of F-PKB and a double asterisk denotes the migration time of phosphorylated F-PKB from cells on the day of the experiment. A) Electrophoretic trace from the CACE analysis of a single HT1080 cell in which virtually all of the substrate was in the phosphorylated form. B) A more representative CACE analysis of an HT1080 cell loaded as in “A”. C) Analysis of an RBL cell loaded with F-PKB-C-SS-TAT (750 nM extracellular concentration). D) Analysis of an RBL cell loaded with F-PKB-C-SS-TAT as in C) and then incubated with PIP₃-histone;

FIG. 7 shows the time course of cleavage of the F-PKB-PLL-TAT. A) In vitro production of free F-PKB after UVA irradiation of a solution of F-PKB-PLL-TAT (1 nM, closed squares; 10 μM, open circles) for varying times. B) Expanded view of graph for times ≦1 min;

FIG. 8 depicts analysis of single cells loaded with peptide substrates conjugated to TAT(49-57) through a photolabile bond. Representative electrophoretic traces from the CACE analyses of cells loaded with F-PKB-PLL-TAT or F-CamKII-PLL-TAT. A single asterisk denotes the migration time of F-PKB or F-CamKII and a double asterisk denotes the migration time of phosphorylated F-PKB or phosphorylated F-CamKII from cells on the day of the experiment. A) Electrophoretic trace from the CACE analysis of a single HT1080/PTEN cell loaded with F-PKB-PLL-TAT (1 μM). B) Electrophoretic trace from the CACE analysis of a single HT1080 cell loaded with F-PKB-PLL-TAT (1 μM). The cells in “A” and “B” were investigated with different capillaries, accounting for the variation in migration times. C) Analysis of an HT1080 cell loaded with F-CamKII-PLL-TAT (750 nM);

FIG. 9 shows the time and temperature dependence of cytosolic entry and accessibility of TAT-loaded cargo. A) Shown is the time dependence of the amount of detectable F-PKB (left axis, squares) and the percentage phosphorylation of that F-PKB (right axis, circles) per HT1080 cell (n=4 for each point). B) Shown is the temperature dependence of the amount of detectable F-PKB (left axis, squares) and the percentage phosphorylation of that F-PKB (right axis, circles) per HT1080 cell (n=4 for each point). Each point reflects the average measurement value and the error bars represent the standard error. The error bars are shown in only one direction for clarity.

### DETAILED DESCRIPTION

The invention solves the problem of breaching the cell membrane to delivery membrane impermeant molecules. The invention includes a method and substance of matter for introducing molecules into cells. The method includes peptide transporters to drive cell-impermeable peptide reporters across cellular membranes.

The invention includes a method for introducing reporters of enzyme activation into cells. The invention includes a peptide that facilitates the transport of molecules (such as molecules which are substrates for enzymes) into a cell (a peptide transduction domain, PTID, or cell-penetrating peptide, CPP) which is chemically conjugated to a reporter of enzyme activation (which includes a substrate peptide and fluorescent tag). Several approaches to conjugation are presented. The resulting molecule can be designed so that the PTD is permanently attached or removed once the reporter molecule is transported into the cell.

The invention enables loading cells with otherwise membrane-impermeable enzyme reporters in a manner that is less stressful to the cell and less labor-intensive for the investigator, while potentially offering greater control over the intracellular release of the reporter. Cells are bathed in solutions of the PTD-conjugated reporter, permitting transport across the cellular membrane. The remaining extracellular peptides are washed away (and, in some cases, the PTD is cleaved), allowing subsequent investigation of intracellular enzymes.

The invention allows for high throughput loading of reporters into cells without physically damaging the cells. The invention may save considerable money in that no additional equipment (such as a microinjector, pulsed laser, or electric field generator) is required and smaller amounts of loading material are typically used. Additionally, the use of PTD-modified reporters does not require specialized skills and can be done by simply exchanging solutions. Further, the invention can potentially be used to exact greater control over the availability of the reporter within the cell. This includes using a photo-cleavable caging mechanism to cleave the compound and release the reporter. Moreover, PTD-mediated loading results in less cellular damage. We have only witnessed damage to cells at very high concentrations.

The PTD can be attached to the reporter molecule by a cleavable or non-cleavable linkage. A cleavable linkage is particularly useful when a covalently attached PTD negatively impacts the interaction of the reporter molecule with the enzyme of interest, or when the properties of the PTD make subsequent analysis of the PTD-reporter conjugate difficult. Representative PID's are depicted on Table 1.

Non-Cleavable PTD Conjugates

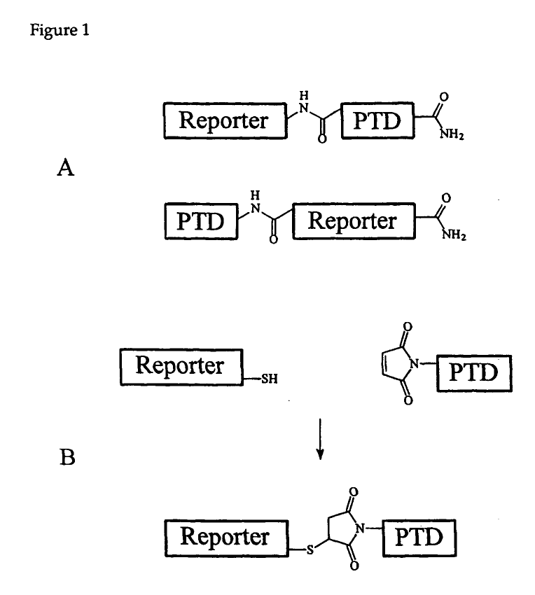

Peptide based reporters are synthesized by solid phase methods with an additional PTD domain at either the C-terminus or N-terminus. If required, linkers can be introduced to separate the PTD from the reporter. If the combined length of the PTD and peptide reporter is too great to be easily prepared by solid phase synthesis, the components can be synthesized separately and ligated through sulfide/maleimide chemistry (see FIG. 1).

Disulfide Cleavable PTD Conjugates

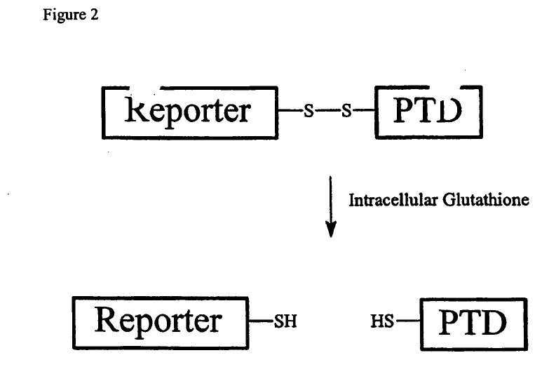

The reporter and PTD can be ligated through disulfide bond. The PTD effects transportation into the cell where the disulfide linkage is cleaved, likey by endogenous glutathione or other cellular components (see FIG. 2).

Photo-Cleavable PTD Conjugates

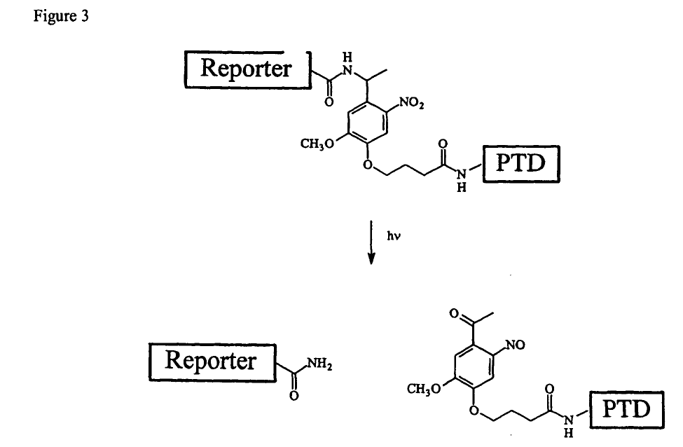

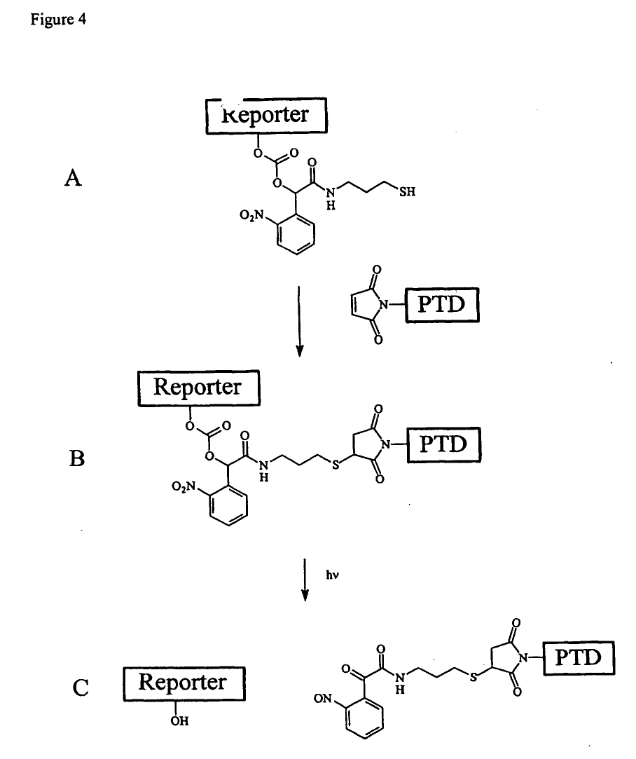

As an example, two methods of attaching a photo-cleavable PTD can be given. In the first, conjugate is prepared by synthesizing the PTD using standard solid phase methods. A photo-cleavable amino acid is then introduced and then the peptide reporter is synthesized. The entire peptide is cleaved from the bead, purified and used. In the second method, one of the amino acids of the reporter is introduced in a form that has been synthetically modified with a sulfide containing photo-cleavable group. The modified amino acid is chosen to be one crucial for the interaction between the reporter and the enzyme. The purified peptide is then conjugated to a maleimide labeled PTD. The resulting molecule will enter the cell, but will likely have reduced, or no, interactions with the enzyme of interest until the PTD is photochemically cleaved (see FIGS. 3 and 4). This method avoids the complication caused by the presence of a PTD-modified reporter with partial activity. Of course, one of skill in the art will be able to introduce these groups and linkages by any available method in the art.

Similarly, even if the PTD is not attached to a crucial amino acid, a photolabile group may be still present on a crucial amino acid to inhibit or prevent reaction with an enzyme. Also similarly, the photolabile group may be attached to an amino acid which is adjacent, or simply near a crucial amino acid, thereby preventing or inhibiting reaction with enzyme by sterically interfering with binding or reaction with the enzyme. An example of a photolabile linkage is one formed from an Fmoc-aminoethyl photolabile linker, such as 4[4(1-(Fmoc-amino)ethyl)-2-methoxy-5-nitrophenoxy] butanoic acid. Other examples of photolabile groups are described by U.S. Pat. No. 5,917,016 titled “PHOTOLABILE COMPOUNDS AND METHODS FOR THEIR USE” to Christopher P. Holmes, which issued on Jun. 29, 1999, and U.S. Pat. No. 6,310,083 titled “Caged amino acid derivatives bearing photolabile protective groups” to Kao, et al. which issued on Oct. 30, 2001.

Biologically active agents (which encompass therapeutic agents) include, but are not limited to metal ions, which are typically delivered as metal chelates; small organic molecules, such as anticancer (e.g., taxane) and antimicrobial molecules (e.g., against bacteria or fungi such as yeast); and macromolecules such as nucleic acids, peptides, proteins, and analogs thereof. In one preferred embodiment, the agent is a nucleic acid or nucleic acid analog, such as a ribozyme which optionally contains one or more 2′-deoxy nucleotide subunits for enhanced stability. Alternatively, the agent is a peptide nucleic acid (PNA). In another preferred embodiment, the agent is a polypeptide, such as a protein antigen, and the biological membrane is a cell membrane of an antigen-presenting cell (APC). In another embodiment, the agent is selected to promote or elicit an immune response against a selected tumor antigen. In another preferred embodiment, the agent is a taxane or taxoid anticancer compound. In another embodiment, the agent is a non-polypeptide agent, preferably a non-polypeptide therapeutic agent. In a more general embodiment, the agent preferably has a molecular weight less than 10 kDa.

In another embodiment, the cleavable linker contains a first cleavable group that is distal to the biologically active agent, and a second cleavable group that is proximal to the agent, such that cleavage of the first cleavable group yields a linker-agent conjugate containing a nucleophilic moiety capable of reacting intramolecularly to cleave the second cleavable group, thereby releasing the agent from the linker and polymer.

In another embodiment, the invention can be used to screen a plurality of conjugates for a selected biological activity, wherein the conjugates are formed from a plurality of candidate agents. The conjugates are contacted with a cell that exhibits a detectable signal upon uptake of the conjugate into the cell, such that the magnitude of the signal is indicative of the efficacy of the conjugate with respect to the selected biological activity. This method is particularly useful for testing the activities of agents that by themselves are unable, or poorly able, to enter cells to manifest biological activity. In one embodiment, the candidate agents are selected from a combinatorial library.

In another aspect, the invention includes a pharmaceutical composition for delivering a biologically active agent across a biological membrane. The composition comprises a conjugate containing a biologically active agent covalently attached to at least one transport polymer as described above, and a pharmaceutically acceptable excipient. The polymer is effective to impart to the agent a rate of trans-membrane transport that is greater than the trans-membrane transport rate of the agent in non-conjugated form. The composition may additionally be packaged with instructions for using it.

In another aspect, the invention includes a therapeutic method for treating a mammalian subject, particularly a human subject, with a pharmaceutical composition as above.

These and other objects and features of the invention will become more fully apparent when the following detailed description of the invention is read in conjunction with the accompanying drawings.

Amino Acids

In one embodiment, the transport polymer is composed of D or L amino acid residues. Use of naturally occurring L-amino acid residues in the transport polymers has the advantage that break-down products should be relatively non-toxic to the cell or organism. Preferred amino acid subunits are arginine (α-amino-δ-guanidinovaleric acid) and α-amino-ε-amidinohexanoic acid (isosteric amidino analog). The guanidinium group in arginine has a pKₐ of about 12.5.

Other amino acids, such as α-amino-β-guanidino-propionic acid, α-amino-γ-guanidinobutyric acid, or α-amino-ε-guanidinocaproic acid can also be used (containing 2, 3 or 5 linker atoms, respectively, between the backbone chain and the central guanidinium carbon).

D-amino acids may also be used in the transport polymers. Compositions containing exclusively D-amino acids have the advantage of decreased enzymatic degradation. However, they may also remain largely intact within the target cell. Such stability is Typically not problematic if the agent is biologically active when the polymer is still attached. For agents that are inactive in conjugate form, a linker that is cleavable at the site of action (e.g., by enzyme- or solvent-mediated cleavage within a cell) should be included within the conjugate to promote release of the agent in cells or organelles.

Other Subunits

Subunits other than amino acids may also be selected for use in forming transport polymers. Such subunits may include, but are not limited to hydroxy amino acids, N-methyl-amino acids, amino aldehydes, and the like, which result in polymers with reduced peptide bonds. Other subunit types can be used, depending on the nature of the selected backbone, as discussed in the next section.

A. Chemical Linkages

Biologically active agents such as small organic molecules and macromoles can be linked to transport polymers of the invention via a number of methods known in the art (see, for example, Wong, 1991), either directly (e.g., with a carbodiimide) or via a linking moiety. In particular, carbamate, ester, thioether, disulfide, and hydrazone linkages are typically easy to form and suitable for most applications. Ester and disulfide linkages are preferred if the linkage is to be readily degraded in the cytosol, after transport of the substance across the cell membrane.

Various functional groups (hydroxyl, amino, halogen, etc.) can be used to attach the biologically active agent to the transport polymer. Groups which are not known to be part of an active site of the biologically active agent are preferred, particularly if the polypeptide or any portion thereof is to remain attached to the substance after delivery.

Polymers, such as peptides produced according to Example 1, are Typically produced with an amino terminal protecting group, such as Fmoc. For biologically active agents which can survive the conditions used to cleave the polypeptide from the synthesis resin and deprotect the side-chains, the Fmoc may be cleaved from the N-terminus of the completed resin-bound polypeptide so that the agent can be linked to the free N-terminal amine. In such cases, the agent to be attached is typically activated by methods well known in the art to produce an active ester or active carbonate moiety effective to form an amide or carbamate linkage, respectively, with the polymer amino group. Of course, other linking chemistries can also be used.

To help minimize side-reactions, guanidino and amidino moieities can be blocked using conventional protecting groups, such as carbobenzyloxy groups (CBZ), di-t-BOC, PMC, Pbf, N-NO₂, and the like.

Coupling reactions are performed by known coupling methods in any of an array of solvents, such as N,N-dimethyl formamide (DMF), N-methyl pyrrolidinone, dichloromethane, water, and the like. Exemplary coupling reagents include O-benzotriazolyloxy tetramethyluronium hexafluorophosphate (HATU), dicyclohexyl carbodiimide, bromo-tris (pyrrolidino) phosphonium bromide (PyBroP), etc. Other reagents can be included, such as N,N-dimethylamino pyridine (DMAP), 4-pyrrolidino pyridine, N-hydroxy succinimide, N-hydroxy benzotriazole, and the like.

For biologically active agents that are inactive until the attached transport polymer is released, the linker is preferably a readily cleavable linker, meaning that it is susceptible to enzymatic or solvent-mediated cleavage in vivo. For this purpose, linkers containing carboxylic acid esters and disulfide bonds are preferred, where the former groups are hydrolyzed enzymatically or chemically, and the latter are severed by disulfide exchange, e.g., in the presence of glutathione.

In one preferred embodiment, the cleavable linker contains a first cleavable group that is distal to the agent, and a second cleavable group that is proximal to the agent, such that cleavage of the first cleavable group yields a linker-agent conjugate containing a nucleophilic moiety capable of reacting intramolecularly to cleave the second cleavable group, thereby releasing the agent from the linker and polymer. This embodiment is further illustrated by the various small molecule conjugates discussed below.

B. Fusion Polypeptides

Transport peptide polymers of the invention can be attached to biologically active polypeptide agents by recombinant means by constructing vectors for fusion proteins comprising the polypeptide of interest and the transport peptide, according to methods well known in the art. Preferably, the transport peptide component will be attached at the C-terminus or N-terminus of the polypeptide of interest, optionally via a short peptide linker.

Enhanced Transport of Biologically Active Agents Across Biological Membranes

A. Measuring Transport Across Biological Membranes

Model systems for assessing the ability of polymers of the invention to transport biomolecules and other therapeutic substances across biological membranes include systems that measure the ability of the polymer to transport a covalently attached fluorescent molecule across the membrane. For example, fluorescein (˜376 MW) can serve as a model for transport of small organic molecules (see Example 2). For transport of macromolecules, a transport polymer can be fused to a large polypeptide such as ovalbumin (molecular weight 45 kDa; e.g., see Example 14). Detecting uptake of macromolecules may be facilitated by attaching a fluorescent tag. Cellular uptake can also be analyzed by confocal microscopy (see Example 4).

B. Enhanced Transport Across Biological Membranes

In experiments carried out in support of the present invention, transmembrane transport and concomitant cellular uptake was assessed by uptake of a transport peptide linked to fluorescein, according to methods described in Examples 2 and 3. Briefly, suspensions of cells were incubated with fluorescent conjugates suspended in buffer for varying times at 37° C., 23° C., or 4° C. After incubation, the reaction was stopped and the cells were collected by centrifugation and analyzed for fluorescence using fluorescence-activated cell sorting (FACS).

Under the conditions used, cellular uptake of the conjugates was not saturable. Consequently, ED₅₀ values could not be calculated for the peptides. Instead, data are presented as histograms to allow direct comparisons of cellular uptake at single conjugate concentrations.

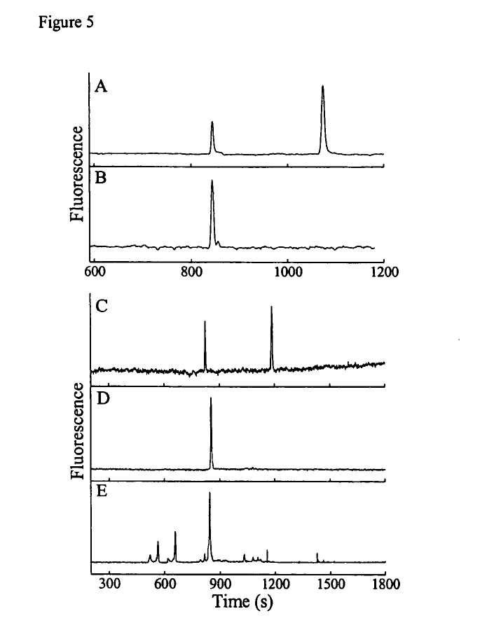

FIGS. 5A-C show results from a study in which polymers of L-arginine (R; FIG. 5A) or D-arginine (r; FIG. 5B) ranging in length from 4 to 9 arginine subunits were tested for ability to transport fluorescein into Jurkat cells. For comparison, transport levels for TAT₍₄₉₋₅₇₎(SEQ ID NO:2) and a nonamer of L-lysine (K9) were also tested. FIG. 5C shows a histogram of uptake levels for the conjugates at a concentration of 12.5 μM.

As shown in the figures, fluorescently labeled peptide polymers composed of 6 or more arginine residues entered cells more efficiently than TAT₍₄₉₋₅₇₎. In particular, uptake was enhanced to at least about twice the uptake level of TAT₍₄₉₋₅₇₎, and as much as about 6-7 times the uptake level of TAT₍₄₉₋₅₇₎. Uptake of fluorescein alone was negligible. Also, the lysine nonamer showed very little uptake, indicating that short lysine polymers are ineffective as trans-membrane transports, in contrast to comparable-length guanidinium-containing polymers.

With reference to FIG. 5B, homopolymers of D-arginine exhibited even greater transport activity than the L-counterparts. However, the order of uptake levels was about the same. For the D-homopolymers, the peptides with 7 to 9 arginines exhibited roughly equal activity. The hexamer (R6 or r6) was somewhat less effective, but still exhibited at least about 2 to 3-fold higher transport activity than TAT₍₄₉₋₅₇₎.

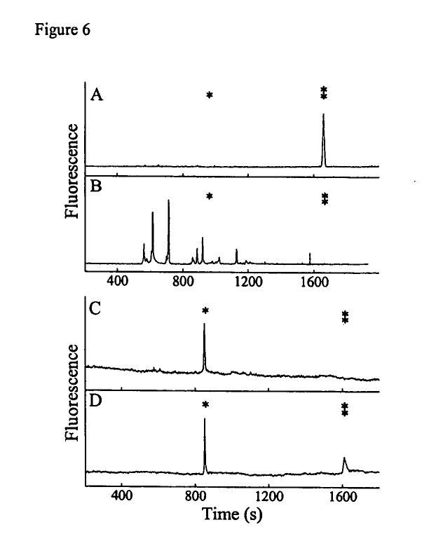

The ability of the D- and L-arginine polymers to enhance trans-membrane transport was confirmed by confocal microscopy (see FIGS. 6A-F and Example 4). Consistent with the FACS data described above, the cytosol of cells incubated with either R9 (see FIGS. 6B and 6E) or r9 (see FIGS. 6C and 6F) was brightly fluorescent, indicating high levels of conjugate transport into the cells. In contrast, TAT₍₄₉₋₅₇₎ at the same concentration showed only weak staining (see FIGS. 6A and 6D). The confocal micrographs also emphasize the point that the D-arginine polymer (see FIG. 6C) was more effective at entering cells than the polymer composed of L-arginine (see FIG. 6F).

From the foregoing, it is apparent that transport polymers of the invention are significantly more effective than TAT₍₄₉₋₅₇₎ in transporting drugs across the plasma membranes of cells. Moreover, the lysine nonamer was ineffective as a transporter.

To determine whether there was an optimal length for contiguous guanidinium-containing homopolymers, a set of longer arginine homopolymer conjugates (R15, R20, R25, and R30) were examined. To examine the effect of substantially longer polymers, a mixture of L-arginine polymers with an average molecular weight of 12,000 daltons (−100 amino acids) was also tested (see Example 5). However, to avoid precipitation problems, the level of serum in the assay had to be reduced for testing conjugates with the .infin.12,000 MW polymer material. Cell uptake was analyzed by FACS as above, and the mean fluorescence of live cells was measured. Cytotoxicity of each conjugate was also measured.

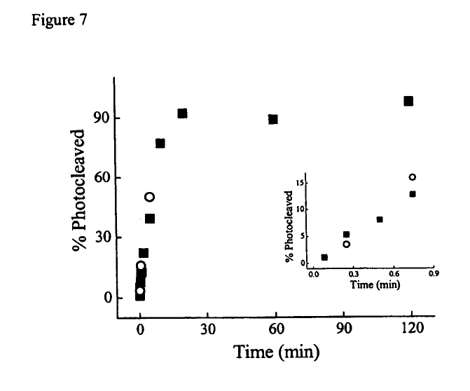

With reference to FIG. 7, uptake of L-arginine homopolymer conjugates with 15 or more arginines exhibited patterns of cellular uptake distinctly different from polymers containing nine arginines or less. The curves of the longer conjugates were flatter, crossing those of the R9 and r9 conjugates. At higher concentrations (>3 μM), uptake of R9 and r9 was significantly better than for the longer polymers. However, at lower concentrations, cells incubated with the longer peptides exhibited greater fluorescence.

Based on this data, it appears that r9 and R9 enter the cells at higher rates than polymers containing 15 or more contiguous arginines. However, the biological half-life of R9 (L-peptide) was shorter than for the longer conjugates, presumably because proteolysis of the longer peptides (due to serum enzymes) produces fragments that retain transport activity. In contrast, the D-isomer (r9) did not show evidence of proteolytic degradation, consistent with the high specificity of serum proteases for L-polypeptides.

Thus, overall transport efficacy of a transport polymer appears to depend on a combination of (i) rate of trans-membrane uptake (polymer with less than about 15 continuous arginines are better) versus susceptibility to proteolytic inactivation (longer polymers are better). Accordingly, polymers containing 7 to 20 contiguous guanidinium residues, and preferably 7 to 15, are preferred.

Notably, the high molecular weight polyarginine conjugate (12,000 MW) did not exhibit detectable uptake. This result is consistent with the observations of Barsoum et al. (1994), and suggests that arginine polymers have transport properties that are significantly different from those that may be exhibited by lysine polymers. Furthermore, the 12,000 polyarginine conjugate was found to be highly toxic (see Example 5). In general, toxicity of the polymers increased with length, though only the 12,000 MW conjugate showed high toxicity at all concentrations tested.

When cellular uptake of polymers of D- and L-arginine were analyzed by Michaelis-Menten kinetics (see Example 6), the rate of uptake by Jurkat cells was so efficient that precise Kₘ values could only be obtained when the assays were carried out at 4° C. (on ice). Both the maximal rate of transport (Vₘₐₓ) and the apparent affinity of the peptides for the putative receptor of the Michaelis constant (Kₘ) were derived from Lineweaver-Burk plots of the observed fluorescence of Jurkat cells after incubation with varying concentrations of nonamers of D- and L-arginine for 30, 60, 120, and 240 seconds.

Kinetic analysis also reveals that polymers rich in arginine exhibit a better ability to bind to and traverse a putative cellular transport site than, for example, TAT₍₄₉₋₅₇₎, since the Kₘ for transport of the nonameric poly-L-arginine (44 μM) was substantially lower than the Kₘ of TAT₍₄₉₋₅₇₎(722 μM). Moreover, the nonamer of D-arginine exhibited the lowest Kₘ (7.21 μM) of the polymers tested in this assay, i.e., an approximately 100-fold greater apparent affinity (see Table 2).

According to a preferred embodiment of the invention, the transport polymer of the invention has an apparent affinity (Kₘ) that is at least 10-fold greater, and preferably at least 100-fold greater, than the affinity measured for tat by the procedure of Example 6 when measured at room temperature (23° C.) or 37° C.

TABLE 2
Kinetic data for peptide binding and transport across membranes

| Peptide | Kₘ (μM) | Vₘₐₓ (μM/sec) |
|---|---|---|
| ⁺H₃N—RRRRRRRRR—CO₂ | 44.43 | 0.35 |
| ⁺H₃N-rrrrrrrrr-CO₂⁻ | 7.21 | 0.39 |
| TAT₍₄₉₋₅₇₎ (SEQ ID NO: 2) | 722 | 0.38 |

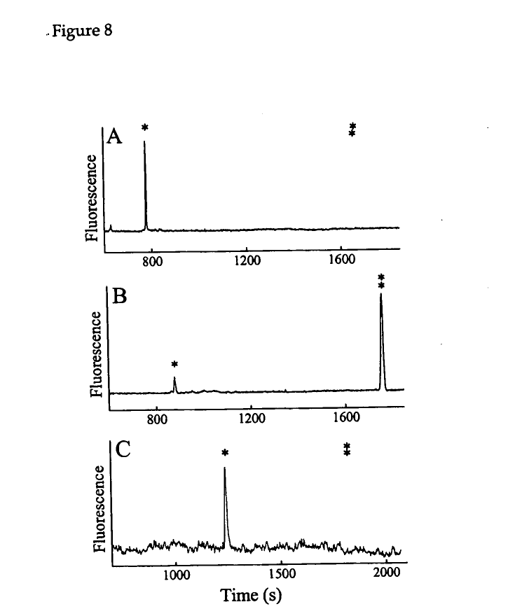

Experiments carried out in support of the present invention indicate that polymer-facilitated transport is dependent upon metabolic integrity of cells. Addition of a toxic amount of sodium azide (0.5% w/v) to cells resulted in inhibition of uptake of conjugates by about 9% (see Example 7). The results shown in FIG. 8 demonstrate (i) sodium azide sensitivity of trans-membrane transport, suggesting energy-dependence (cellular uptake), and (ii) the superiority of poly-guanidinium polymers of the invention (R9, R8, R7) relative to TAT₍₄₉₋₅₇₎.

Without ascribing to any particular theory, the data suggest that the transport process is an energy-dependent process mediated by specific recognition of guanidinium or amidinium-containing polymers by a molecular transporter present in cellular plasma membranes.

Other experiments in support of the invention have shown that the conjugates of the invention are effective to transport biologically active agents across membranes of a variety of cell types, including human T cells (Jurkat), B cells (murine CH27), lymphoma T cells (murine EL-4), mastocytoma cells (murine P388), several murine T cell hybridomas, neuronal cells (PC-12), fibroblasts (murine RT), kidney cells (murine HELA), myeloblastoma (murine K562); and primary tissue cells, including all human blood cells (except red blood cells), such as T and B lymphocytes, macrophages, dendritic cells, and eosinophils; basophiles, mast cells, endothelial cells, cardiac tissue cells, liver cells, spleen cells, lymph node cells, and keratinocytes.

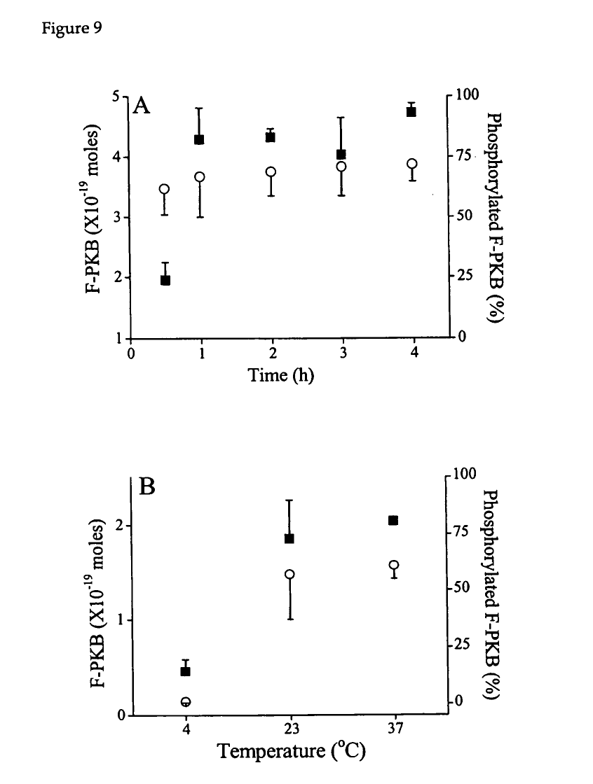

The conjugates are also effective to traverse both gram negative and gram positive bacterial cells, as disclosed in Example 8 and FIGS. 9A-C. In general, polymers of D-arginine subunits were found to enter both gram-positive and gram-negative bacteria at rates significantly faster than the transport rates observed for polymers of L-arginine. This is illustrated by FIG. 9A, which shows much higher uptake levels for r9 conjugate (D-arginines), than for the R9 conjugate (L-arginines), when incubated with E. coli HB101 (prokariotic) cells. This observation may be attributable to proteolytic degradation of the L-polymers by bacterial enzymes.

FIG. 9B shows uptake levels for D-arginine conjugates as a function of length (r4 to r9) in comparison to a poly-L-lysine conjugate (19), when incubated with E. coli HB101 cells. As can be seen, the polyarginine conjugates showed a trend similar to that in FIG. 6B observed with eukariotic cells, such that polymers shorter than r6 showed low uptake levels, with uptake levels increasing as a function of length.

Gram-positive bacteria, as exemplified by Strep. bovis, were also stained efficiently with polymers of arginine, but not lysine, as shown in FIG. 9C.

More generally, maximum uptake levels by the bacteria were observed at 37° C. However, significant staining was observed when incubation was performed either at room temperature or at 4° C. Confocal microscopy revealed that pretreatment of the bacteria with 0.5% sodium azide inhibited transport across the inner plasma membranes of both gram-positive and gram-negative bacteria, but not transport across the cell wall (gram-positive bacteria) into the periplasmic space.

Thus, the invention includes conjugates that contain antimicrobial agents, such as antibacterial and antifungal compounds, for use in preventing or inhibiting microbial proliferation or infection, and for disinfecting surfaces to improve medical safety. In addition, the invention can be used for transport into plant cells, particularly in green leafy plants.

Additional studies in support of the invention have shown that translocation across bacterial membranes is both energy- and temperature-dependent, consistent with observations noted earlier for other cell-types.

Definitions

The term “biological membrane” as used herein refers to a lipid-containing barrier which separates cells or groups of cells from the extracellular space. Biological membranes include, but are not limited to, plasma membranes, cell walls, intracellular organelle membranes, such as the mitochondrial membrane, nuclear membranes, and the like.

The term “transmembrane concentration” refers to the concentration of a compound present on the side of a membrane that is opposite or “trans” to the side of the membrane to which a particular composition has been added. For example, when a compound is added to the extracellular fluid of a cell, the amount of the compound measured subsequently inside the cell is the transmembrane concentration of the compound.

“Biologically active agent” or “biologically active substance” refers to a chemical substance, such as a small molecule, macromolecule, or metal ion, that causes an observable change in the structure, function, or composition of a cell upon uptake by the cell. Observable changes include increased or decreased expression of one or more mRNAs, increased or decreased expression of one or more proteins, phosphorylation of a protein or other cell component, inhibition or activation of an enzyme, reaction with an enzyme, inhibition or activation of binding between members of a binding pair, an increased or decreased rate of synthesis of a metabolite, increased or decreased cell proliferation, and the like.

The term “macromolecule” as used herein refers to large molecules (MW greater than 1000 daltons) exemplified by, but not limited to, peptides, proteins, oligonucleotides and polynucleotides of biological or synthetic origin.

“Small organic molecule” refers to a carbon-containing agent having a molecular weight (MW) of less than or equal to 1000 daltons.

The terms “therapeutic agent”, “therapeutic composition”, and “therapeutic substance” refer, without limitation, to any composition that can be used to the benefit of a mammalian species, particularly humans. Such agents may take the form of ions, small organic molecules, peptides, proteins or polypeptides, oligonucleotides, and oligosaccharides, for example.

The terms “agricultural agent”, “agricultural composition”, and “agricultural substance” refer, without limitation, to any composition that can be used to the benefit of a plant species. Such agents may take the form of ions, small organic molecules, peptides, proteins or polypeptides, oligonucleotides, and oligosaccarides, for example.

The terms “non-polypeptide agent” and “non-polypeptide therapeutic agent” refer to the portion of a transport polymer conjugate that does not include the transport-enhancing polymer, and that is a biologically active agent other than a polypeptide. An example of a non-polypeptide agent is an anti-sense oligonucleotide, which can be conjugated to a poly-arginine peptide to form a conjugate for enhanced delivery across biological membranes.

The term “polymer” refers to a linear chain of two or more identical or non-identical subunits joined by covalent bonds. A peptide is an example of a polymer that can be composed of identical or non-identical amino acid subunits that are joined by peptide linkages.

The term “peptide” as used herein refers to a compound made up of a single chain of D- or L-amino acids or a mixture of D- and L-amino acids joined by peptide bonds. Preferably, peptides contain at least two amino acid residues and are less than about 50 amino acids in length.

The term “protein” as used herein refers to a compound that is composed of linearly arranged amino acids linked by peptide bonds, but in contrast to peptides, has a well-defined conformation. Proteins, as opposed to peptides, Preferably contain chains of 50 or more amino acids.

“Polypeptide” as used herein refers to a polymer of at least two amino acid residues and which contains one or more peptide bonds. “Polypeptide” encompasses peptides and proteins, regardless of whether the polypeptide has a well-defined conformation.

The terms “guanidyl,” “guanidinyl,” and “guanidino” are used interchangeably to refer to a moiety having the formula HN═C(NH₂)NH— (unprotonated form). As an example, arginine contains a guanidyl (guanidino) moiety, and is also referred to as 2-amino-5-guanidinovaleric acid or α-amino-δ-guanidinovaleric acid. “Guanidinium” refers to the positively charged conjugate acid form.

“Amidinyl” and “amidino” refer to a moiety having the formula —C(═NH)(NH₂). “Amidinium” refers to the positively charged conjugate acid form.

The term “poly-arginine” or “poly-Arg” refers to a polymeric sequence composed of contiguous arginine residues; poly-L-arginine refers to all L-arginines; poly-D-arginine refers to all D-arginines. Poly-L-arginine is also abbreviated by an upper case “R” followed by the number of L-arginines in the peptide (e.g., R8 represents an 8-mer of contiguous L-arginine residues); poly-D-arginine is abbreviated by a lower case “r” followed by the number of D-arginines in the peptide (r8 represents an 8-mer of contiguous D-arginine residues).

Amino acid residues are referred to herein by their standard single-letter or three-letter notations or by their full names: A, Ala, alanine; C, Cys, cysteine; D, Asp, aspartic acid; E, Glu, glutamic acid; F, Phe, phenylalanine; G, Gly, glycine; H, His, histidine; I, Ile, isoleucine; K, Lys, lysine; L, Leu, leucine; M, Met, methionine; N, Asn, asparagine; P, Pro, proline; Q, Gln, glutamine; R, Arg, arginine; S, Ser, serine; T, Thr, threonine; V, Val, valine; W, Trp, tryptophan; X, Hyp, hydroxyproline; Y, Tyr, tyrosine.

The term “peptide conjugate” refers to any peptide that is attached to another molecule, such as a label, another peptide, a polypeptide, an amino acid, a polysaccharide, a nucleic acid, etc.

The term “membrane traversing peptide conjugate” means that some membrane must be available for which the peptide conjugate is capable of traversing.

The term “target peptide” refers to a peptide as a reactant for a chemical reaction occurring in a cell, such as an enzyme-catalyzed reaction, a nucleic acid binding reaction, a polysaccharide binding reaction, a polypeptide binding reaction, etc.

The term “label” refers to any moiety that is capable of detection, selection, or amplification, such as a radioactive element, a fluorescent moiety, a phosphorescent moiety, a luminescent moiety, a chemiluminescent moiety, metal coordination group (for example, a group that becomes fluorescent after metal or ion coordinates), an epitope for an antibody (which may be detected by reaction with a fluorescently labeled antibody), etc.

The term “transduction domain” has the same meaning as described herein for peptide transduction domain, or cell-penetrating peptide.

The term “chimera” refers to a non-naturally occurring cell derived from more than one naturally occurring cell, such as mouse-human hybrids, hybridomas, etc.

The term “cargo” refers to a molecule, such as peptide, polynucleotide, ligand, or an enzyme substrate that is initially attached to the transduction domain for delivery across a biological membrane.

The phrase “PTD-cargo conjugate” refers to a molecule containing a peptide transduction domain attached to a cargo molecule though at least one covalent bond.

The term “enzyme substrate” refers to a substrate for an enzyme-catalyzed reaction. Typical substrates include, but are not limited to, polypeptides, nucleic acids, polysaccharides, lipids, small organic molecules, macromolecules, biologically active agents, therapeutically active agents, agriculturally active agents, etc. Typical enzymes include proteins and nucleic acids, such as catalytic DNA molecules, catalytic RNA molecules or ribozymes.

The term “photolabile linkage” refers to a site of covalent attachment that is susceptible to cleavage following irradiation with light. An example of a photolabile linkage is one formed from an Fmoc-aminoethyl photolabile linker, such as 4[4-(1-(Fmoc-amino)ethyl)-2-methoxy-5-nitrophenoxy] butanoic acid. Other examples of photolabile linkages are described by U.S. Pat. No. 5,917,016 titled “PHOTOLABILE COMPOUNDS AND METHODS FOR THEIR USE” to Christopher P. Holmes, which issued on Jun. 29, 1999.

Utility

Beyond those methods disclosed herein, the present invention may be adapted for use with the instrument designed to perform high throughput analysis of multiple cellular samples using the machine disclosed in U.S. Pat. No. 6,335,201, titled “METHOD AND APPARATUS FOR DETECTING ENZYMATIC ACTIVITY USING MOLECULES THAT CHANGE ELECTROPHORETIC MOBILITY” to Allbritton et al., which issued on Jan. 1, 2002.

### EXAMPLES

The following examples are presented to aid practitioners of the invention, provide experimental support for the invention, and to provide model protocols. In no way are these examples to be understood to limit the invention.

### Example 1

### Characterization of TAT₍₄₉₋₅₇₎ Conjugated to a Substrate Peptide

To determine whether a kinase substrate linked to TAT₍₄₉₋₅₇₎ was accessible to cytosolic enzymes, rat basophilic leukemia (RBL) cells were incubated with 1 μM calcium-calmodulin dependent kinase II peptide substrate (F-CamKII; SEQ ID NO:11) conjugated to TAT₍₄₉₋₅₇₎(SEQ ID NO:2) through a peptide bond (F-CamKII-TAT; SEQ ID NO:12). To determine if F-CamKII-TAT (SEQ ID NO:12) is phosphorylated by its target kinase, CamKII, cells were loaded with F-CamKII-TAT and then their contents analyzed for phosphorylated F-CamKII using the Cell Activity by Capillary Electrophoresis (CACE) (also known as LMS or Laser Micropipet System, see Methods, below). With the CACE, a single cell can be rapidly lysed and the cellular contents loaded with nearly 100% efficiency into a capillary exceptionally quickly (33×10⁻³ seconds) (Sims, Meredith et al. 1998; Meredith, Sims et al. 2000; Li, Sims et al. 2001). Together with cell lysis, electrophoresis is initiated to separate cellular contents. The fast lysis coupled with turbulent mixing and electrophoretic separation terminate the cellular reactions almost immediately (Meredith, Sims et al. 2000; Li, Sims et al. 2001). Until the instant of lysis, however, the cell rests undisturbed and is bathed in physiologic buffer. Phosphorylated and non-phosphorylated forms of a kinase-substrate peptide are separated by electrophoresis, which are then detected by laser-induced fluorescence. This analysis provides a “snapshot” of kinase activity at the time of cell lysis, eliminating common artifacts such as spontaneous kinase or phosphatase activity, which may occur in traditional methods. Table I summarizes the experimental design.

TABLE I
Experimental design

| Sample | Exogenous | |
|---|---|---|
| set | polypeptide | Notes |
| Control | None | Measures any background activity |
| 1 | (=untreated cells) | detected by CACE. Any observed |
| | | signal here is subtracted from the |
| | | signal in the experimental samples. |
| Control | F-CamKII | Unconjugated CamKII peptide |
| 2 | (SEQ ID NO: 11) | substrate is usually barred from |
| | | cellular entry by the cell membrane. |
| | | This sample establishes the basal rate |
| | | of F-CamKII entry into the cell. |
| Experi- | F-CamKII-TAT | Conjugated CamKII peptide substrate. |
| mental | (SEQ ID NO: 12) | If conjugate is phosphorylated |
| | | (asindicated by laser-induced |
| | | fluorescence in the CACE), then this |
| | | observation suggests that it has |
| | | breached the cell membrane. |

No fluorescent peaks were observed in untreated, single cells, even after 6000 seconds. When F-CamKII alone (i.e., without being conjugated to TAT₍₄₉₋₅₇₎was loaded into cells by microinjection, and the cells were analyzed using the CACE, F-CamKII possessed a migration time of 800 seconds and a detection limit of 3×10⁻²¹ moles.

Since F-CamKII-TAT (SEQ ID NO:12) has a higher net positive charge-to-mass ratio than the unconjugated F-CamKII (SEQ ID NO:11), F-CamKII-TAT was expected to have an electrophoretic migration time much shorter than 800 seconds. To determine whether the TAT₍₄₉₋₅₇₎ sequence might be causing anomalous electrophoretic behavior of F-CamKII-TAT, standard solutions of fluorescein-TAT₍₄₉₋₅₇₎ and F-CamKII-TAT were loaded into capillaries and electrophoresis initiated. Neither peptide eluted from the capillary within 6000 seconds, even when 10⁻¹⁷ moles was loaded. When the F-CamKII-TAT was placed on a glass surface similar to the fused-silica inner walls of the capillary, the peptide strongly adsorbed to the glass surface. The most likely explanation for the absence of a detectable fluorescent peak during electrophoresis was the adsorption of the highly positive TAT₍₄₉₋₅₇₎ sequence to the negatively-charged glass wall of the capillary.

In addition to assessing the electrophoretic behavior of F-CamKII-TAT, the ability of this conjugated sequence to act as an efficient substrate for the CamKII enzyme was characterized. The amount of phosphate incorporated into the substrate peptide alone and into F-CamKII-TAT by CamKII was measured in vitro. In the range of measured peptide concentrations, 90% less phosphate was incorporated by the CamKII enzyme into F-CamKII-TAT compared to the peptide lacking the TAT₍₄₉₋₅₇₎ sequence. The kinetic properties of CamKII enzyme for the CamKII peptide substrate were degraded when the peptide was conjugated to TAT₍₄₉₋₅₇₎.

### Example 2

### Characterization of Substrate Peptides Conjugated to TAT₍₄₉₋₅₇₎ by a Disulfide Bond

Previous investigators developed cleavable PTD-cargo conjugates by engineering a disulfide linkage between the two domains (Stein, Weiss et al. 1999; Hallbrink, Floren et al. 2001). In the oxidative extracellular environment, the disulfide bond remained intact, but in the reductive intracellular environment, the disulfide bond quickly reduced releasing the cargo from the PTD (Meister 1989; Hallbrink, Floren et al. 2001). TAT₍₄₉₋₅₇₎(SEQ ID NO:2) was conjugated via a disulfide bond to cysteines added to substrate peptides for CamKII (F-CamKII-C; SEQ ID NO:13) or PKB (F-PKB-C; SEQ ID NO:14), denoted F-CamKII-C-SS-TAT (SEQ ID NO:15) and F-PKB-C-SS-TAT (SEQ ID NO:16), respectively. The TAT₍₄₉₋₅₇₎-Conjugated substrates were incubated with cells (HT1080 or HT1080/PTEN cells) and the contents of the cells were analyzed by the CACE. HT1080 cells have constitutive PKB activity due to a mutated allele of the N-ras gene, resulting in the subsequent activation of signaling proteins downstream of the ras protein (Gupta, Plattner et al. 2000). A second cell line, HT1080/PTEN, has been engineered to over-express wild type PTEN, a lipid phosphatase which metabolizes phosphatidylinositol 3,4,5-trisphosphate (PIP3), resulting in greatly diminished PKB activity in the HT1080/PTEN cells (Gupta and Stanbridge 2001). HT1080 cells do not have constitutive CamKII enzyme activity.

Electrophoretic traces of the contents of all HT1080 cells loaded with F-CamKII-C-SS-TAT (SEQ ID NO:15) at an extracellular concentration of 750 nM demonstrated a single major peak with a migration time identical to that of a standard of F-CamKII-C (SEQ ID NO:13) (n>12). This is seen in FIG. 5A, showing the electrophoretic trace of a mixture of F-CamKII (9×10⁻²⁰ mol) and phosphorylated F-CamKII (5×10¹⁹ mol) in buffer solution, and FIG. 5B, depicting a typical electrophoretic trace of a single HT1080 cell loaded with the disulfide conjugate F-CamKII-C-SS-TAT. Thus, the disulfide bond between the F-CamKII-C and TAT₍₄₉₋₅₇₎ portions of SEQ ID NO:15 was successfully reduced upon entry into the cells, releasing the substrate cargo. As expected, F-CamKII-C alone (SEQ ID NO:13) showed little to no phosphorylation in the HT1080 cells since no peaks or only very small peaks appeared at the migration time for the phosphorylated form of the peptide.

In contrast, electrophoretic traces from HT1080/PTEN cells loaded with F-PKB-C-SS-TAT (1 μM extracellular concentration) demonstrated a single peak in only 30% of the cells analyzed (n=19). For this subpopulation of cells, the migration time of the single peak was identical to that of a standard of F-PKB-C (FIG. 5C, showing the electrophoretic trace of a mixture of F-PKB (8×10⁻²⁰ mol) and phosphorylated F-PKB (1×10⁻¹⁹ mol) in buffer solution; and FIG. 5D, depicting an electrophoretic trace of a single HT1080/PTEN cell loaded with the disulfide conjugate F-PKB-C-SS-TAT at an extracellular concentration of 1 μM). As expected, no peak was present that migrated at the time of the phosphorylated form of F-PKB-C (SEQ ID NO:14) in cells, since PKB has little to no activity in the HT1080/PTEN cells. In the remaining 70% of the cells, multiple peaks were present on the electrophoretic trace. One of the major peaks possessed a migration time identical to that of a standard of F-PKB-C (SEQ ID NO:14; FIG. 5E). Of the other peaks, none possessed a migration time consistent with phosphorylated F-PKB-C. Since TAT₍₄₉₋₅₇₎'s adsorption to the capillary surface prevented migration through the capillary, the additional peaks could not be F-PKB-C still conjugated to TAT₍₄₉₋₅₇₎. It was hypothesized that these peaks were the result of oxidative reactions of the sulfhydryl group present on the F-PKB-C peptide. To determine whether the unidentified peaks could be converted to F-PKB-C by reduction of the sulfhydryl, HT1080/PTEN cells were loaded with F-PKB-C-SS-TAT (SEQ ID NO:16) as above, and the reducing agent DTT (1 μM) was added to the solution surrounding the cells just prior to lysis and analysis by the CACE. DTT was also added to the electrophoretic buffer contained in the capillary. Whenever DTT was present, a single peak with a migration time identical to that of F-PKB-C was present on the electrophoretic trace. These data suggest that the additional peaks arose from intracellular reactions of the sulfhydryl group on the peptide.

### Example 3

### Phosphorylation of Substrate Peptides Delivered by a Disulfide-Linked TAT₍₄₉₋₅₇₎

To determine whether the detached peptide substrates were in the cytosol and accessible to kinases, the intracellular phosphorylation of the translocated cargo was studied. In the initial experiment, F-CamKII-C-SS-TAT (SEQ ID NO:15) at 750 nM extracellular concentration was loaded into HT1080 cells. The cells were incubated with ionomycin (500 nM) for 15 min to increase the intracellular concentration of free Ca²⁺ and activate the CamKII enzyme (Worrell and Frizzell 1991). The electrophoretic traces from these cells displayed a second peak in addition to the peak of due to the non-phosphorylated F-CamKII-C. The migration time of the second peak was identical to that of phosphorylated F-CamKII-C loaded into cells. 52+3% (mean±SEM) (n=5) of the F-CamKII-C was phosphorylated as determined by comparison of the peak areas. At least half of the F-CamKII-C was in the cytosol and accessible to the kinase. For these cells the average number of moles per cell was ˜2×10⁻²⁰ which is equivalent to a cytosolic concentration of ˜100 nM.

Since the properties of F-PKB (SEQ ID NO:17) microinjected into HT1080 cells have been well characterized, experiments were undertaken to compare the fate of F-PKB-C loaded as F-PKB-C-SS-TAT (SEQ ID NO:16) into HT1080 cells to that of the microinjected F-PKB (SEQ ID NO:17). When microinjected, the percentage of phosphorylated F-PKB depended on the relative, steady-state levels of both the kinases and phosphatases. This observation is shown in electrophoretic traces from cells loaded with F-PKB-C-SS-TAT (1 μM) (FIG. 6). A single asterisk denotes the migration time of F-PKB, and a double asterisk denotes the migration time of phosphorylated F-PKB from cells on the day of the experiment. FIG. 6A depicts the electrophoretic trace from the CACE analysis of a single HT1080 cell in which virtually all of the substrate was in the phosphorylated form, while FIG. 6B shows a more representative CACE analysis of an HT1080 cell loaded as in FIG. 6A.

At total concentrations of F-PKB (SEQ ID NO:17) below the Kₘ of PKB and the known Kₘ's of the phosphatases for their substrates, the percent of phosphorylated F-PKB was independent of the total concentration of peptide. In HT1080 cells, approximately 70% of the peptide was phosphorylated at steady state which was achieved in less than 2 min. In addition, all of the peptide loaded by microinjection was shown to be accessible to PKB for phosphorylation. When HT1080 cells were incubated with F-PKB-C-SS-TAT (SEQ ID NO:16), 85% (n=85) of the electrophoretic traces possessed multiple peaks with migration times other than that of non-phosphorylated and phosphorylated F-PKB-C (SEQ ID NO:14; FIG. 6B). Addition of DTT to the media surrounding these cells at the time of lysis, converted the migration times of these peaks to that of non-phosphorylated F-PKB-C. These oxidized forms of F-PKB-C were either not accessible to PKB or not good substrates for PKB. In a minority of cells (15%, n=15), only two peaks were present on the electrophoretic trace, and the migration times of these peaks were identical to that of non-phosphorylated and phosphorylated F-PKB-C. The percentage peptide present as phosphorylated F-PKB-C in these cells was 75±8%. This percentage of phosphorylated F-PKB-C was nearly identical to that measured when HT1080 cells were microinjected with F-PKB, suggesting that all of the detected F-PKB-C was fully accessible to PKB in this subfraction of cells. The amount of peptide detected with the CACE was quantified by comparison to standards, and on average each cell possessed 7×10⁻¹⁹±0.3×10⁻¹⁹ moles of detectable F-PKB-C. This represents a cytosolic concentration of ˜700 nM free F-PKB-C.

If an easily saturated step was present during the separation from TAT and reduction of the substrate, the inability of many HT1080 cells to maintain the F-PKB-C in its reduced form might have been due to the relatively high concentrations of F-PKB-C in the cell. For this reason HT1080 cells were incubated in an extracellular concentration of 215 nM F-PKB-C-SS-TAT. At this lower concentration, 67% of the cells (n=6) demonstrated electropherograms with only non-phosphorylated and phosphorylated F-PKB-C and no other peaks. The average amount of detected F-PKB-C in this subfraction of cells was 2.5×10⁻¹⁹±0.3×10⁻¹⁹ moles. This represents a cytosolic concentration of ˜250 nM free F-PKB-C. To determine whether other cell types might also have a limited capacity to reduce the disulfide bond in F-PKB-C-SS-TAT, RBL cells were loaded with F-PKB-C-SS-TAT (SEQ ID NO:16). All cells (n=3) loaded at an extracellular concentration of 750 nM showed a peak with the same migration time as F-PKB-C and no additional peaks. These cells contained 1.4×10⁻²⁰ ±0.1×10⁻²⁰ moles of detectable F-PKB-C which represents an intracellular concentration of ˜14 nM. In contrast, when the extracellular concentration was increased to 3 μM, all RBL cells (n=4) showed a multitude of peaks most of which did not possess migration times of non-phosphorylated or phosphorylated F-PKB-C. While achieving full reduction of F-PKB-C-SS-TAT (SEQ ID NO:16) was concentration dependent, HT1080 and RBL cells loaded with F-CamKII-C-SS-TAT (SEQ ID NO:15) were rarely seen to demonstrate unexpected peaks regardless of the loading concentration used (<1 in 50 cells for an extracellular concentration range of 100 nM-1 μM). Thus, F-CamKII-C (SEQ ID NO:13) appeared to be more easily maintained in the reduced state compared to F-PKB-C (SEQ ID NO:14).

To determine whether the F-PKB-C delivered to cells by addition of F-PKB-C-SS-TAT (SEQ ID NO:16) at a 750 nM extracellular concentration could also be phosphorylated after pharmacologic activation of PKB (rather than constitutively active PKB), PIP3-histone was added to RBL cells. Exogenous phosphoinositides delivered to cells using cationic carriers, such as histone, have been demonstrated to activate PKB and exert downstream physiological responses (Wang, Herzmark et al. 2002; Weiner, Neilsen et al. 2002). The CACE was used to analyze the amount of phosphorylated F-PKB-C in the presence (15 μM) and absence of PIP3-histone added for 10 min to RBL cells loaded with F-PKB-C-SS-TAT (FIG. 6C,D). In RBL cells exposed to PIP3-histone, 39%±14% (n=4) of F-PKB-C was phosphorylated while no phosphorylation was present in unexposed cells (FIG. 6D).

Design of a Photo-Cleavable Substrate Peptide-TAT₍₄₉₋₅₇₎ Conjugate.

While substrate peptides disulfide-linked to TAT₍₄₉₋₅₇₎ could in some instances be delivered to the cytosol in a fully reduced form accessible to the intended kinases, this was not always the case. Therefore, we designed a second detachable linker between the TAT₍₄₉₋₅₇₎ and the substrate peptide. An Fmoc-aminoethyl photolabile moiety was inserted between the fluorescently-labeled substrate and the TAT₍₄₉₋₅₇₎ sequences using standard Fmoc peptide chemistry. The relative order of the different portions of the conjugate was: fluorescein-substrate-PLL-TAT₍₄₉₋₅₇₎. Two different substrate peptides were conjugated to TAT₍₄₉₋₅₇₎ to form F-PKB-PLL-TAT (SEQ ID NO:18) and F-CamKII-PLL-TAT (SEQ ID NO:19). To determine the time and irradiation intensity needed to photocleave the conjugated peptides, standard solutions (1 nM or 10 μM) of F-PKB-PLL-TAT (SEQ ID NO:18) were illuminated with long-wavelength UV light (UVA) for varying times. The sample was then loaded into a capillary and electrophoresis performed. The concentration of the released substrate peptide was quantified from its fluorescence. At the irradiation intensity used, maximal cleavage of the substrate peptide from the TAT₍₄₉₋₅₇₎ sequence was achieved within 20 minutes. These results are shown in FIG. 7. FIG. 7A shows in vitro production of free F-PKB after UVA irradiation (1 nM, closed squares; 10 μM, open circles) for varying times; FIG. 7B expands the view of the graph for times ≦1 min. After a 10 s exposure, 2% of the conjugate was photoreleased. Without UVA illumination, no free substrate peptide could be detected in electrophoretic separations. The fluorescein was not substantially photobleached by the UVA-induced cleavage since the amount of fluorescent peptide detected after a 4 h irradiation was almost identical to that after 20 min of UVA exposure.

### Example 4

### Characterization of Photo-Cleavable Substrate Peptide-TAT₍₄₉₋₅₇₎ Conjugates in Cells

HT1080/PTEN cells were loaded with F-PKB-PLL-TAT (SEQ ID NO:18) at 1 μm extracellular concentration in a manner identical to the loading of the disulfide conjugates. After loading, the cells were illuminated with UVA light for 10 s, washed, and then incubated an additional 10 min. Single cells were then selected for CACE analysis. In contrast to the disulfide conjugate, a single peak with a migration time identical to that of a standard of F-PKB was the only peak present on the electrophoretic trace in all cells analyzed (n=6). FIGS. 8A,B show typical traces of such an experiment using a single HT1080/PTEN cell loaded with F-PKB-PLL-TAT (1 μM). While the F-PKB-PLL-TAT was taken into the cell and a portion of the F-PKB was successfully detached from the TAT₍₄₉₋₅₇₎ sequence, it was unlikely that all of the F-PKB was photo-released due to the very short irradiation time. The measured F-PKB represents only the photocleaved portion of the conjugate since the intact F-PKB-PLL-TAT could not be detected due to its adsorption onto the capillary walls as discussed above. As expected, phosphorylation of the released F-PKB was not seen in HT1080/PTEN cells. To determine whether a substrate peptide other than F-PKB could also be successfully loaded into cells as a photolabile conjugate, HT1080 cells were incubated with F-CamKII-PLL-TAT (SEQ ID NO:19; 100 nM). When this conjugate was photocleaved in the cells, a single peak with a migration time identical to that of the F-CamKII (SEQ ID NO:11) standard was present on the electropherogram (FIG. 8C).

### Example 5

### Phosphorylation of Substrate Peptides Delivered by a Photo-Cleavable TAT₍₄₉₋₅₇₎

HT1080 cells were loaded with F-PKB-PLL-TAT (SEQ ID NO:18) at 1 μM extracellular concentration in an identical manner to that of the HT1080/PTEN cells above. When the contents of the HT1080 cells analyzed with the CACE, two peaks were typically present on the electrophoretic trace, one with the migration time of F-PKB and one with the migration time of phosphorylated F-PKB (FIG. 8B). No other peaks were present on the electrophoretic trace. 67±7% (n=15) of the F-PKB was present in the phosphorylated form. These results were nearly identical to those measured when F-PKB was microinjected or was loaded successfully with the disulfide conjugate.

To determine whether a substrate peptide for the CamKII enzyme could be phosphorylated when loaded into cells as a photolabile conjugate, HT1080 cells were incubated with F-CamKII-PLL-TAT (SEQ ID NO:19) at a 300 nM extracellular concentration. Ionomycin (500 nM) was then added to the cells and the cells were incubated for 15 min. Following lysis and electrophoresis, two peaks were present on the electrophoretic trace of the cell's contents. The migration times of the peaks were similar to that of the non-phosphorylated and phosphorylated standards of F-CamKII (SEQ ID NO:11) loaded into cells. On average 67±17% (n=3) of the total F-CamKII was phosphorylated.

To determine the total amount of free F-PKB released into the cytoplasm and detectable by the CACE, HT1080 cells were incubated with F-PKB-PLL-TAT (SEQ ID NO:18) (1 μM) for 5 min then washed. After a 30 min incubation, cells were UVA-irradiated for 10 s, allowed to recover for 10 min, and analyzed with the CACE. The average quantity of F-PKB detected per cell was 2×10⁻¹⁹±0.9×10⁻¹⁹ moles (n=15). This value represents an approximate cytoplasmic concentration of 200 nM of free F-PKB. When HT1080 cells loaded with F-CamKII-PLL-TAT (SEQ ID NO:19) at a 300 nM extracellular concentration, each cell contained 1.8×10⁻¹⁹±1.9×10⁻¹⁹ moles (n=3) of detectable F-CamKII. This represents an average concentration of 180 nM F-CamKII in the cytosol of the cells.

### Example 6

### Time Dependence of Cytosolic Entry and Accessibility of TAT-Loaded Cargo

Prior work suggests that the initial step in TAT-mediated import of cargo into a cell is the adsorption of TAT to the surface of the cell presumably via an electrostatic interaction (Schwarze, Hruska et al. 2000; Wadia and Dowdy 2002). The next step involving internalization of the TAT-cargo into the cell appears to be a much slower process and may be mediated by endocytosis or another mechanism. Recent work by Fittapaldi et al. demonstrated that for intact, folded proteins this second step required as long as 10 h to reach completion (Fittipaldi, Ferrari et al. 2003). This result contrasts with other studies that measured very fast uptake times for TAT and its cargo (Wender, Mitchell et al. 2000; Hallbrink, Floren et al. 2001; Ho, Schwarze et al. 2001). One possible explanation is that the different methods used might detect TAT at different steps during its uptake into a cell. For this reason, we measured the time required for TAT-mediated delivery of F-PKB to the cytosol and the time required for F-PKB to become accessible to PKB after adsorption of F-PKB-PLL-TAT (SEQ ID NO:18) to the plasma membrane. HT1080 cells were incubated with F-PKB-PLL-TAT (SEQ ID NO:18) at a 1 μM extracellular concentration at 37° C. for 5 min to adsorb TAT to the cell surface. The cells were washed removing all F-PKB-PLL-TAT dissolved in the media, and were then incubated for varying times at 37° C. After this incubation time, the cells were irradiated for 10 s with UVA light and analyzed with the CACE. The total amount of F-PKB detected with the CACE increased over the initial hour of incubation and then stabilized. These results are depicted in FIG. 9A, showing the time dependence of the amount of detectable F-PKB (left axis, squares) and the percentage phosphorylation of that F-PKB (right axis, circles) per HT1080 cell (n=4 for each point). In FIG. 9B, the temperature dependence of the amount of detectable F-PKB (left axis, squares) and the percentage phosphorylation of that F-PKB (right axis, circles) per HT1080 cell (n=4 for each point) is shown. Each point reflects the average measurement value and the error bars represent the standard error. The error bars are shown in only one direction for clarity. The percentage of phosphorylated F-PKB was similar to that measured previously in HT1080 cells and did not vary with the incubation time (FIG. 9A). These results suggest that for short peptide cargoes, the internalization process into the cytosol is complete within one hour, and the F-PKB is accessible to kinase on time scales much faster 30 min.

### Example 7

### Temperature Dependence of Cytosolic Entry and Accessibility of TAT-Loaded Cargo

Previous experiments measuring the temperature dependence of TAT-mediated translocation has yielded discrepant results. Some of these differences may be due to TAT adsorption to the surface of the cell which occurs at all temperatures whereas actual internalization may be more heavily dependent on temperature (Richard, Melikov et al. 2003). Adsorbed TAT that was loaded into the cell after removal of the soluble TAT might then be indistinguishable from that loaded into the cell during the time the cell was incubated with soluble TAT. HT1080 cells were loaded with F-PKB-PLL-TAT (SEQ ID NO:18) at a 1 μM extracellular concentration as described in the “Experimental Procedures” except that the loading and incubation steps were performed at either 4° C., 23° C., or 37° C. The cells were then immediately analyzed with the CACE while still in the solution at 4° C., 23° C., or 37° C. The amount of F-PKB measured per cell was similar at both 23° C. and 37° C., but was reduced in cells maintained at 4° C. (see FIG. 9B). Loading does occur at low temperature, although less efficiently than at higher temperatures. Since the percentage of phosphorylated F-PKB was similar at 23° C. and 37° C., the cargo was readily available for phosphorylation when released from TAT at these temperatures. Essentially no phosphorylation occurred at 4° C. (see FIG. 9B). This was most likely due to decreased kinase activity rather than to inaccessible F-PKB, although the two possibilities cannot be distinguished from these experiments.

### Example 8

### Experimental Procedures for Examples 1-7

Materials

Tissue culture reagents were purchased from Invitrogen (Grand Island, N.Y.). [□-³²P]-ATP was purchased from New England Nuclear (Boston, Mass.). Extracellular buffer (ECB) was composed of 135 mM NaCl, 5 mM KCl, 10 mM HEPES (pH 7.4), 1 mM MgCl₂, 1 mM CaCl₂. Capillaries were purchased from Polymicro Technologies (Phoenix, Ariz.). Bovine serum albumin (BSA) was purchased from Sigma-Aldrich (St. Louis, Mo.). N-succinimidyl 3-[2-pyridyldithio]-propionamido (SPDP) was obtained from Molecular Biosciences, Inc. (Boulder, Colo.). Recombinant calcium-calmodulin activated kinase II (CamKII) and calmodulin were purchased from Calbiochem (San Diego, Calif.). D-myo-phosphatidylinositol 3,4,5-trisphosphate and D(+)-sn-1,2-di-o-hexadecanoylglyceryl, 3-O-phospho linked (DiC16PtdIns[3,4,5]P3) and Histone H1 were a kind gift from Echelon (Salt Lake City, Utah). All other reagents were purchased from Fisher Scientific (Pittsburg, Pa.).

Peptides and Peptide Components

Fmoc-protected amino acids and Fmoc-aminoethyl photolabile linker (4-[4-(1-(Fmoc-amino)ethyl)-2-methoxy-5-nitrophenoxy] butanoic acid) were purchased from Novabiochem (Switzerland). The peptides shown in Table II were synthesized with amidated carboxy termini and then fluorescently labeled and purified at the Beckman Peptide and Nucleic Acid Facility at Stanford University (Stanford, Calif.), unless indicated otherwise. The phosphorylated forms (phosphates on the underlined residues) were also synthesized at the Beckman Peptide and Nucleic Acid Facility with amino termini then fluorescently labeled and purified. F-CamKII (SEQ ID NO:11), a substrate for CamKII, was derived from the threonine 286 autophosphorylation site on CamKII (Hanson, Kapiloff et al. 1989). F-PKB (SEQ ID NO:17), a substrate for protein kinase B (PKB), was based on the substrate RPRAATF as originally described by Alessi et al (Alessi, Caudwell et al. 1996).

F-PKB-C-SS-TAT and F-PKB-PLL-TAT (SEQ ID NOs:16 and 18, respectively) were synthesized and purified “in house” as described below. F-CamKII-C-SS-TAT and F-CamKII-PLL-TAT and (SEQ ID NOs:15 and 19, respectively) were synthesized and purified at Anaspec (San Jose, Calif.).

Cell Culture

Human sarcoma HT-1080 and rat basophilic leukemia (RBL-2H3) cells were cultured in Dulbecco's modified Eagle medium (DMEM; e.g., from Invitrogen; Carlsbad, Calif.) supplemented with 10% fetal calf serum (FCS), 4 mM L-glutamine, 100 U/mL penicillin and 100 μg/mL streptomycin at 37° C. in 5% CO₂. HT-1080/PTEN cells were maintained in the same medium with the addition of a selective antibiotic (800 μg/ml of Geneticin).

Cells used in experiments were grown in a cell chamber made by using Sylgard 184 (Dow Corning; Midland, Mich.) to attach a Teflon “O ” ring ( 15/16 in outer diameter) to a 25 mm, round, # 1 glass cover slip. In some case, the cover slips were coated with collagen to increase cellular adherence to the glass surface. Cells were plated at a density of 5-10 cells per high power field. Prior to use, the cells were cultured for 12-24 hours after plating in the cell chamber. Cells were serum-starved 18 hours prior to experiments, using 0.25% FCS DMEM. Experiments with cells were conducted at 37° C. unless otherwise indicated.

TABLE II
Summary of polypeptides

| | | |
|---|---|---|
| | | SEQ |
| | | ID |
| Polypeptide | Sequence | NO: |

| | | |
|---|---|---|
| F¹-CamKII | Lys Lys Ala Leu His Arg Gln Glu Thr Val Asp Ala Leu | 11 |
| | 1 5 10 | |
| F¹-CamKII-C | Lys Lys Ala Leu His Arg Gln Glu Thr Val Asp Ala Leu Cys | 13 |
| | 1 5 10 | |
| F¹-PKB | Gly Arg Pro Arg Ala Ala Thr Phe Ala Glu Gly | 17 |
| | 1 5 10 | |
| F¹-PKB-C | Gly Arg Pro Arg Ala Ala Thr Phe Ala Glu Gly Cys | 14 |
| | 1 5 10 | |
| TAT₍₄₉₋₅₇₎ | Arg Lys Lys Arg Arg Gln Arg Arg Arg | 2 |
| | 1 5 | |
| F¹-CamKII- | Lys Lys Ala Leu His Arg Gln Glu Thr Val Asp Ala Leu Arg Lys Lys Arg Arg Gln Arg Arg Arg | 12 |
| TAT | 1 5 10 15 20 | |
| F¹-CamKII- | Lys Lys Ala Leu His Arg Gln Glu Thr Val Asp Ala Leu Cys Arg Lys Lys Arg Arg Gln Arg Arg | 15 |
| C-SS³-TAT | 1 5 10 15 20 | |
| | Arg | |
| | 23 | |
| F¹-PKB-C- | Gly Arg Pro Arg Ala Ala Thr Phe Ala Glu Gly Cys Arg Lys Lys Arg Arg Gln Arg Arg Arg | 16 |
| SS³-TAT | 1 5 10 15 20 | |
| F¹-PKB- | Gly Arg Pro Arg Ala Ala Thr Phe Ala Glu Gly Arg Lys Lys Arg Arg Gln Arg Arg Arg | 18 |
| PLL²-TAT | 1 5 10 15 20 | |
| F¹-CamKII- | Lys Lys Ala Leu His Arg Gln Glu Thr Val Asp Ala Leu Cys Arg Lys Lys Arg Arg Gln Arg Arg | 19 |
| PLL²-TAT | 1 5 10 15 20 | |
| | Arg | |
| | 23 | |

¹F, fluorescein
²PLL, photolabile linker; for SEQ ID NOs: 18 and 19, PLL is 4-[4-(1-aminoethyl)-2-methoxy-5-nitrophenoxy] butanoic acid.
³SS, disulfide-bond linkage between the peptides as indicated

Labeling Peptides with Fluorescein; Peptide Purification and Characterization

Peptides were labeled with fluorescein at the amino termini, and de-protected as previously described (Sims, Meredith et al. 1998). Peptides were purified by high-pressure liquid chromatography (HPLC) as previously described (Meredith, Sims et al. 2000). Molecular weight and peptide purity were assessed by Matrix Assisted Laser Desorption Ionization Mass Spectrometry (MALDI-MS; Voyager System 4124; Applied Biosystems; Foster City, Calif.) and capillary electrophoresis (CE), respectively. Fluorescein-labeled peptide concentrations were determined by amino acid analysis at the Molecular Structure Facility at the University of California, Davis by adding to the sample a standard of known concentration.

Peptide Conjugation Through a Disulfide Bond

TAT₍₄₉₋₅₇₎, attached to the synthetic resin, was modified on its amino-terminus with 50 μmoles of the hetero-bifunctional cross-linker N-Succinimidyl-3 (2-PyridylDithio)-Propionate (SPDP) (or a 1:5 molar ratio (TAT₍₄₉₋₅₇₎:SPDP)) in 256 μL of dichloromethane in the presence of 1.2 equivalents of di-isopropylethylamine for 2 hours. The pyridyldithiol group preferentially reacts with free sulfhydryls, limiting the generation of disulfide-linked TAT₍₄₉₋₅₇₎ homodimers (e.g., TAT₍₄₉₋₅₇₎-SS-TAT₍₄₉₋₅₇₎) (Pain and Surolia 1981). The peptide was then cleaved in 95% trifluoroacetic acid (v/v), 2.5% water (v/v), 2.5% anisole (v/v), ether-precipitated, and dissolved in a minimal volume of 100 mM triethylammonium acetate (pH 7). The SPDP-reacted TAT₍₄₉₋₅₇₎ peptide was added at an estimated molar ratio of 1:1 to the fluorescein-labeled substrate, which contained a cysteine on the carboxy terminus. The mixture was incubated at room temperature (20-22° C.) for 2 hours.

Peptide Conjugation Through a Photolabile Linker

The Fmoc-aminoethyl photolabile linker was added to the amino-terminus of resin-bound TAT(49-57) using standard Fmoc (N-(9-fluorenyl)methoxycarbonyl) chemistry at 0.1 mmole scale on an ABI model 433A peptide synthesizer (Applied Biosystems). The PKB substrate peptide (SEQ ID NO:17) was then added to the photolabile linker using the synthesizer. Fluorescein was added to the amino terminus of the resin-bound peptide and the peptide was de-protected and purified.

In Vitro Kinase Assay

Peptides (5-60 μM) were phosphorylated in vitro by 100 units of recombinant CamKII (EMD Biosciences; San Diego, Calif.) in 20 mM Tris-HCl, 10 mM MgCl₂, 0.5 mM dithiothreitol (DTT), and 0.1 mM Ethylenediaminetetraacetic acid-(EDTA, pH 7.5) with 2.4 mM calmodulin, 2 mM CaCl₂ and 100 mM [□-³²P]-ATP. The final specific activity of the [□-³²P]-ATP was 100 cpm/pmole; the total reaction volume was 25 μL. The mixture was incubated at 30° C. for 30 minutes. The reaction was stopped by adding 45 μL ice-cold 10% (v/v) trichloroacetic acid, mixed with a vortex, centrifuged, and blotted onto P81 phosphocellulose paper. Phosphate incorporation was quantified with a liquid scintillation counter (LS 3801; Beckman Instruments; Irvine, Calif.)

Cell Activity by Capillary Electrophoresis (CACE)

CACE has been previously described (Sims, Meredith et al. 1998; Meredith, Sims et al. 2000; Li, Wu et al. 2001). Briefly, cells were placed on an inverted fluorescence microscope stage (Diaphot 300, Nikon, Japan) and maintained at 37° C. with an objective heater (Bioptechs; Butler, Pa.) prior to lysis unless stated otherwise. The cells were continually washed with ECB maintained at 37° C. (unless otherwise indicated) with a flow rate of 1 ml/min and a total chamber volume of 0.5 ml. The inlet of a capillary (30 μm I.D., 360 μm O.D., 75 cm long) was positioned 10 μm above the cell. The inverted microscope was coupled to a pulsed Nd:YAG laser and a CE system (Sims, 1998). The CACE-based assay was performed as previously described (U, 2001; Meredith, 2000). Cells were always maintained in ECB when analyzed by the CACE.

Capillary Electrophoresis (CE).

CE was performed as described (Sims, Meredith et al. 1998), with the following modifications. The inner walls of the capillaries (30 μm I.D., 360 μm O.D.) were coated with poly(acrylate) (Wang, Hu et al. 2003). The outlet of the capillary was held at a negative potential of 18-21 kV, and the inlet reservoir was held at ground potential. Under these conditions, the current through the capillary was typically ˜36 μA. Solutions of standards were loaded into the capillary by gravitational fluid flow. The loaded volume was calculated from Poiseulle's equation and from contributions by spontaneous fluid displacement and diffusion (Weinberger, 2000; Fishman, 1994; Crank, 1989). To estimate the number of moles of peptide in an unknown solution or within a cell, the peak area from the electrophoretic trace of the solution or the cell was compared to that of a standard of known concentration separated by electrophoresis on the same day. A 1 pL cell volume and 100% peptide recovery was assumed in calculating intracellular peptide concentration (Meredith, Sims et al. 2000; Sims, Meredith et al. 1998). Analytes were detected by laser-induced fluorescence (Sims, Meredith et al. 1998).

Cleavage of the Photolabile Peptides

The photolabile TAT₍₄₉₋₅₇₎ conjugate (e.g., SEQ ID NOs:18 or 19) (1 nM or 10 μM in ECB) was illuminated with ultraviolet A (UVA; 20 mW/cm²) using a 365 nm light source (UVP Blak-Ray B100AP; Upland, Calif.). Emission maxima for the photolabile group was 365 nm. After photo-release, the fluorescent substrate was resolved by electrophoresis and then quantified by CE. Detection wavelengths were: excitation, 488 nm; emission, 505-565 nm.

Construction of Standard Curve for Photolabile Peptides

A known concentration of the photo-cleavable reporter was placed in an uncapped, shallow vial. The sample was then illuminated with the UV light source for varying times as described above. A small amount of the sample was then removed and the amount of released peptide was quantified using CE. The amount of released substrate was plotted, and the linear portion of the curve was then fitted to a straight line using a linear regression algorithm (Origin software; Origin Lab Corp.; North Hampton, Mass.). The slope of this line represents the rate of photo-cleavage.

Loading Cells with TAT₍₄₉₋₅₇₎-Conjugated Substrates

Cells were incubated at room temperature for 5 minutes in 1% bovine serum albumin (BSA) in ECB, washed in ECB, and then incubated for 5 minutes at room temperature with a substrate conjugated to TAT₍₄₉₋₅₇₎. The cells were then washed with 1% BSA in ECB. Cells loaded with the disulfide conjugate were placed in supplement DMEM (10% FCS, 4 mM L-glutamine, 100 U/mL penicillin and 100 μg/mL streptomycin) and incubated at 37° C. in 5% CO₂ for 20 minutes. Cells loaded with the photolabile conjugate were placed in supplemented DMEM and incubated at 37° C. in 5% CO₂ for 10 minutes (unless stated otherwise) and were then illuminated with a UV light source for 10 seconds (a radiant energy/unit area of 2 kJ/m²). The supplemented DMEM was exchanged and the cells were incubated another 10 minutes at 37° C. in 5% CO₂. Just prior to analysis the cells were washed into ECB.

Addition of PIP₃ to Cells

Long chain synthetic phospholipids dipalmitoyl phosphatidylinositol 3,4,5-triphosphate sodium salt (DiC₁₆PtdIns (3,4,5)P₃ sodium salt) or phosphatidylinositol (3,4,5) trisphosphate (PIP3) were freshly prepared at 300 μM in 150 mM NaCl, 4 mM KCl and 20 mM HEPES at pH 7.2 and resuspended by bath sonication or vigorous mixing with a vortex (Weiner, Neilsen et al. 2002). Histone-phospholipid complexes were prepared by combining 300 μM phospholipids with 100 μM freshly prepared histone. The mixture was mixed with a vortex, and then incubated for 5 minutes at room temperature. The solution was diluted 1:10 with ECB immediately before adding to cells. Cells were incubated with the histone-phospholipid complexes for 10 minutes at 37° C. before analysis with the CACE.

### Prophetic Example: Assay of the Effect of a Compound on the Activity of an Enzyme

A membrane traversing peptide is prepared which includes a label, such as a fluorescent molecule, attached to a target peptide, such as a peptide substrate for a kinase, which is in turn attached to a peptide transduction domain, for example by a photolabile linkage. A cell, such as a human cell, in then exposed to the membrane traversing peptide, causing the cell to take it up. Next, the cell is exposed to the compound, such as a drug candidate. The cell is then exposed to light to cleave the photolabile linkage; this activates the target peptide for reaction with the kinase. Then the activity of the kinase is examined, for example by laser lysis of the cell and subsequent electrophoresis, to determine the ratio of unreacted target peptide to reacted target peptide. If the activity of the kinase is changed when the cell is exposed to the compound, this may be attributed to the effects of the compound. In this prophetic example, the cell could have been exposed to the compound before introducing the membrane traversing peptide.

### REFERENCES

Alessi, D. R., F. B. Caudwell, et al. (1996). “Molecular basis for the substrate specificity of protein kinase B; comparison with MAPKAP kinase-1 and p70 S6 kinase.” FEBS Letters 399: 333-338.
Astriab-Fisher, A., D. Sergueev, et al. (2002). “Conjugates of antisense oligonucleotides with the Tat and antennapedia cell-penetrating peptides: effects on cellular uptake, binding to target sequences, and biologic actions.” Pharmaceutical Research 19: 744-754.
Becker-Hapak, M., S. S. McAllister, et al. (2001). “TAT-mediated protein transduction into mammalian cells.” Methods 24(3): 247-56.
Cao, G., W. Pei, et al. (2002). “In Vivo Delivery of a Bcl-xL Fusion Protein Containing the TAT Protein Transduction Domain Protects against Ischemic Brain Injury and Neuronal Apoptosis.” J Neurosci 22(13): 5423-31.
Chen, W.-C. et al. (1978). “Conjugation of Poly-L-Lysine Albumin and Horseradish Peroxidase: A Novel Method of Enhancing the Cellular Uptake of Proteins.” Proc. Natl. Acad. Sci. USA 1975: 1872-76.
Derossi, D., S. Calvet, et al. (1996). “Cell internalization of the third helix of the Antennapedia homeodomain is receptor-independent.” Journal of Biological Chemistry 271:18188-18193.
Fittipaldi, A., A. Ferrari, et al. (2003). “Cell membrane lipid rafts mediate caveolar endocytosis of HIV-1 tat fusion proteins.” J Biol Chem.
Frankel, A. D. and C. O. Pabo (1988). “Cellular uptake of the tat protein from human immunodeficiency virus.” Cell 55(6): 1189-93.
Futaki S., T. Suzuki, et al. (2001). “Arginine-rich peptides. An abundant source of membrane-permeable peptides having potential as carriers for intracellular protein delivery.” J Biol Chem 276(8): 5836-40.
Green, M. and P. M. Loewenstein (1988). “Autonomous functional domains of chemically synthesized human immunodeficiency virus tat trans-activator protein.” Cell 55(6): 1179-88.
Gupta, S., R. Plattner, et al. (2000). “Dissection of Ras-dependent signaling pathways controlling aggressive tumor growth of human fibrosarcoma cells: evidence for a potential novel pathway.” Mol Cell Biol 20(24): 9294-306.
Gupta, S. and E. J. Stanbridge (2001). “Paired human fibrosarcoma cell lines that possess or lack endogenous mutant N-ras alleles as experimental model for Ras signaling pathways.” Methods Enzymol 333: 290-306.
Hallbrink, M., A. Floren, et al. (2001). “Cargo delivery kinetics of cell-penetrating peptides.” Biochim Biophys Acta 1515(2): 101-9.
Hanson, P. I., M. S. Kapiloff, et al. (1989). “Expression of a multifunctional Ca2+/calmodulin-dependent protein kinase and mutational analysis of its autoregulation.” Neuron 3: 59-70.
Ho, A., S. R. Schwarze, et al. (2001). “Synthetic protein transduction domains: enhanced transduction potential in vitro and in vivo.” Cancer Res 61(2): 474-7.
Hoffmann, P. (1988). “Stimulation of Human and Murine Adherent Cells by Bacterial Lipoprotein and Synthetic Lipopeptide Analogues.” Immunobiol. 177: 158-70.
Kelemen, B. R., K. Hsiao, et al. (2002). “Selective in vivo inhibition of mitogen-activated protein kinase activation using cell-permeable peptides.” J Biol Chem 277: 8741-8748.
Leamon, C. P. and P. S. Low (1991). “Delivery of macromolecules into living cells: a method that exploits folate receptor endocytosis.” Proc. Natl. Acad. Sci. U.S.A. 88: 5572-76.
Lewin, M., N. Carlesso, et al. (2000). “Tat peptide-derivatized magnetic nanoparticles allow in vivo tracking and recovery of progenitor cells.” Nat Biotechnol 18(4): 410-4.
Li, H., C. E. Sims, et al. (2001). “Spatial control of cellular measurements with the laser micropipet.” Anal Chem 73(19): 4625-31.
Li, H., H. Y. Wu, et al. (2001). “Improved capillary electrophoresis conditions for the separation of kinase substrates by the laser micropipet system.” J Chromatogr B Biomed Sci Appl 757(1): 79-88.
Lindgren, M., M. Hallbrink, et al. (2000). “Cell-penetrating peptides.” Trends Pharmacol Sci 21(3): 99-103.
Lindsay, M. A. (2002). “Peptide-mediated cell delivery: application in protein target validation.” Curr Opin Pharmacol 2(5): 587-94.
Lipinski, C. A., F. Lombardo, et al. (2001). “Experimental and computational approaches to estimate solubility and permeability in drug discovery and development settings.” Adv Drug Deliv Rev 46(1-3): 3-26.
Meister, A. (1989). Metabolism and function of glutathione. Glutathione: chemical, biochemical and medical aspects. D. Dolphin, R. Poulson and O. Avramovic. New York, Wiley: 367 474.
Meredith, G. D., C. E. Sims, et al. (2000). “Measurement of kinase activation in single mammalian cells.” Nat Biotechnol 18(3): 309-12.
Pain, D. and A. Surolia (1981). “Preparation of protein A-peroxidase monoconjugate using a heterobifunctional reagent, and its use in enzyme immunoassays.” J Immunol Methods 40(2): 219-30.
Prior, T. I. et al. (1991). “Barnase Toxin: A New Chimeric Toxin Composed of Pseudomonas Exotoxin A and Barnase.” Cell 64: 1027-23.
Ribeiro, M. M., D. Klein, et al. (2003). “Heme oxygenase-1 fused to a TAT peptide transduces and protects pancreatic beta-cells.” Biochem Biophys Res Commun 305(4): 876-81.
Richard, J. P., K. Melikov, et al. (2003). “Cell-penetrating peptides. A reevaluation of the mechanism of cellular uptake.” J Biol Chem 278(1): 585-90.
Rothbard, J. B. and P. A. Wender (2001). Method and composition for enhancing transport across biological membranes. U.S. Pat. No. 6,306,993 (Oct. 23, 2001).
Ryser, H. and W.-C. Shen (1989). Method of effecting cellular uptake of molecules. U.S. Pat. No. 4,847,240 (Jul. 11, 1989).
Schwarze, S. R., A. Ho, et al. (1999). “In vivo protein transduction: delivery of a biologically active protein into the mouse.” Science 285(5433): 1569-72.
Schwarze, S. R., K. A. Hruska, et al. (2000). “Protein transduction: unrestricted delivery into all cells?” Trends in Cell Biology 10: 290-295.
Shibagaki, N. and M. C. Udey (2003). “Dendritic cells transduced with TAT protein transduction domain-containing tyrosinase-related protein 2 vaccinate against murine melanoma.” Eur J Immunol 33(4): 850-60.
Sims, C. E., G. D. Meredith, et al. (1998). “Laser-micropipet combination for single-cell analysis.” Analytical Chemistry 70: 4570-7.
Stein, S., A. Weiss, et al. (1999). “A disulfide conjugate between anti-tetanus antibodies and HIV (37-72)Tat neutralizes tetanus toxin inside chromaffin cells.” FEBS Lett 458(3): 383-6.
Sternson, L. A. (1987). Obstacles to Polypeptide Delivery.
Stolzenberger, S., M. Haake, et al. (2001). “Specific inhibition of interleukin-4-dependent Stat6 activation by an intracellularly delivered peptide.” Eur J Biochem 268(17): 4809-14.
Suzuki, T., S. Futaki, et al. (2002). “Possible existence of common internalization mechanisms among arginine-rich peptides.” J Biol Chem 277(4): 2437-43.
Vives, E., P. Brodin, et al. (1997). “A truncated HIV-1 Tat protein basic domain rapidly translocates through the plasma membrane and accumulates in the cell nucleus.” J Biol Chem 272(25): 16010-7.
Vogel, B. E., S. J. Lee, et al. (1993). “A novel integrin specificity exemplified by binding of the alpha v beta 5 integrin to the basic domain of the HIV Tat protein and vitronectin” J Cell Biol 121(2): 461-8.
Wadia, J. S. and S. F. Dowdy (2002). “Protein transduction technology.” Current Opinion in Biotechnology 13: 52-56.
Wang, F., P. Herzmark, et al. (2002). “Lipid products of PI(3)Ks maintain persistent cell polarity and directed motility in neutrophils.” Nature Cell Biology 4(7): 513-518.
Wang, Y., S. Hu, et al. (2003). “Separation of mixtures of acidic and basic peptides at neutral pH.” Journal of Chromatography A 1004: 61-70.
Weiner, O. D., P. O. Neilsen, et al. (2002). “A PtdInsP3- and Rho GTPase-mediated positive feedback loop regulates neutrophil polarity.” Nature Cell Biology 4(7): 503-513.
Wender, P. A., D. J. Mitchell, et al. (2000). “The design, synthesis, and evaluation of molecules that enable or enhance cellular uptake: peptoid molecular transporters.” Proc Natl Acad Sci U S A 97(24): 13003-8.
Worrell, R. T. and R. A. Frizzell (1991). “CaMKII mediated stimulation of chloride conductance by calcium in T84 cells.” Am J Physiol 260: C877-C882.

1. A membrane traversing peptide conjugate, comprising:
(i) a label,
(ii) a target peptide, attached to the label, and
(iii) a transduction domain, attached to the target peptide, wherein the target peptide is a reactant for a chemical reaction occurring in a cell.

2. The membrane traversing peptide conjugate of claim 1, wherein the chemical reaction is catalyzed by an enzyme.

3. The membrane traversing peptide conjugate of claim 2, wherein the enzyme is a kinase.

4. The membrane traversing peptide conjugate of claim 2, wherein the cell is a mammalian cell.

5. The membrane traversing peptide conjugate of claim 4, wherein the mammalian cell is a human cell.

6. The membrane traversing peptide conjugate of claim 2, wherein the cell is at least one member selected from the group consisting of bacteria, insect, and plant cells.

7. The membrane traversing peptide conjugate of claim 2, wherein the cell is a chimera.

8. The membrane traversing peptide conjugate of claim 2, wherein the transduction domain is a member selected from the group consisting of SEQ ID NO:2, SEQ ID NO:3, SEQ ID NO:4, SEQ ID NO:5, SEQ ID NO:6, SEQ ID NO:7, SEQ ID NO:8, and portions thereof.

9. The membrane traversing peptide conjugate of claim 2, wherein the transduction domain is attached to the target peptide by a member selected from the group consisting of a disulfide bond, a photolabile linkage and an ester linkage.

10. The membrane traversing peptide conjugate of claim 2, wherein the label is a member selected from the group consisting of a radioactive element, a fluorescent moiety, a phosphorescent moiety, a luminescent moiety, and a chemiluminescent moiety.

11. A membrane traversing peptide conjugate, comprising:
(i) a label,
(ii) a target peptide, attached to the label, and
(iii) a transduction domain, attached to the target peptide, wherein the target peptide is a reactant for a chemical reaction occurring in a cell,
wherein the label is fluorescent,
wherein the target peptide is a kinase substrate, and
wherein the cell is a human cell.

12. The membrane traversing peptide conjugate of claim 11, wherein the transduction domain is attached to the target peptide by a member selected from the group consisting of a disulfide bond, a photolabile linkage and an ester linkage.

13. The membrane traversing peptide conjugate of claim 11, wherein the kinase substrate is a member selected from the group consisting of SEQ ID NO:11, SEQ ID NO:13, SEQ ID NO:14, and SEQ ID NO:17.

14. The membrane traversing peptide conjugate of claim 11, wherein the transduction domain is SEQ ID NO:2.

15. The membrane traversing peptide conjugate of claim 12, wherein the transduction domain is attached to the target peptide by a disulfide bond.

16. The membrane traversing peptide conjugate of claim 12, wherein the transduction domain is attached to the target peptide by 4-(4-(1-(amino)ethyl)-2-methoxy-5-nitrophenoxy] butanoic acid.

17. The membrane traversing peptide conjugate of claim 11, wherein the label is fluorescein.

18. A membrane traversing peptide conjugate selected from the group consisting of SEQ ID NO:15, SEQ ID NO:16, SEQ ID NO:18 and SEQ ID NO:19.

19-37. (canceled)

38. A membrane traversing peptide conjugate, comprising:
(i) a label,
(ii) an enzyme substrate, attached to the label, and
(iii) a transduction domain, attached to the enzyme substrate, wherein the label is a member selected from the group consisting of a radioactive element, a fluorescent moiety, a phosphorescent moiety, a luminescent moiety, and a chemiluminescent moiety.

39-45. (canceled)

46. A method of measuring the activity of a reaction in a cell, comprising: introducing into the cell a membrane traversing peptide conjugate; and detecting a label optically;
wherein the membrane traversing peptide conjugate comprises:
(i) the label,
(ii) a biologically active agent, attached to the label, and
(iii) a transduction domain, attached to the biologically active agent.

47-50. (canceled)


SEQUENCE LISTING (20 sequences)

SEQ ID NO: 1 - skipped

SEQ ID NO: 2 - protein, 9 aa, Human immunodeficiency virus type 1
RKKRRQRRR

SEQ ID NO: 3 - protein, 16 aa, Unknown Organism
  Description of Unknown Organism: Penetratin sequence
RQIKIWFQNRRMKWKK

SEQ ID NO: 4 - protein, 13 aa, Human immunodeficiency virus type 1
GRKKRRQRRRPPQ

SEQ ID NO: 5 - protein, 27 aa, Human immunodeficiency virus type 1
GALFLGWLGAAGSTMGAWSQPKKKRKV

SEQ ID NO: 6 - protein, 16 aa, Unknown Organism
  Description of Unknown Organism: Signal- sequence-based peptide (II)
AAVALLPAVLLALLAP

SEQ ID NO: 7 - protein, 26 aa, Unknown Organism
  Description of Unknown Organism: Transportan sequence
GWTLNSAGYLLKINLKALAALAKKIL

SEQ ID NO: 8 - protein, 18 aa, Unknown Organism
  Description of Unknown Organism: Amphiphilic model peptide
KLALKLALKALKAALKLA

SEQ ID NO: 9-10 - skipped (2 entries)

SEQ ID NO: 11 - protein, 13 aa, Artificial Sequence
  Description of Artificial Sequence: Synthetic polypeptide
KKALHRQETVDAL

SEQ ID NO: 12 - protein, 22 aa, Artificial Sequence
  Description of Artificial Sequence: Synthetic polypeptide
KKALHRQETVDALRKKRRQRRR

SEQ ID NO: 13 - protein, 14 aa, Artificial Sequence
  Description of Artificial Sequence: Synthetic polypeptide
KKALHRQETVDALC

SEQ ID NO: 14 - protein, 12 aa, Artificial Sequence
  Description of Artificial Sequence: Synthetic polypeptide
GRPRAATFAEGC

SEQ ID NO: 15 - protein, 23 aa, Artificial Sequence
  Description of Artificial Sequence: Synthetic polypeptide
KKALHRQETVDALCRKKRRQRRR

SEQ ID NO: 16 - protein, 21 aa, Artificial Sequence
  Description of Artificial Sequence: Synthetic polypeptide
GRPRAATFAEGCRKKRRQRRR

SEQ ID NO: 17 - protein, 11 aa, Artificial Sequence
  Description of Artificial Sequence: Synthetic polypeptide
GRPRAATFAEG

SEQ ID NO: 18 - protein, 20 aa, Artificial Sequence
  Description of Artificial Sequence: Synthetic polypeptide
GRPRAATFAEGRKKRRQRRR

SEQ ID NO: 19 - protein, 23 aa, Artificial Sequence
  Description of Artificial Sequence: Synthetic polypeptide
KKALHRQETVDALCRKKRRQRRR

SEQ ID NO: 20 - protein, 9 aa, Artificial Sequence
  Description of Artificial Sequence: Synthetic peptide
RRRRRRRRR